(19) United States
(12) Patent Application Publication
(10) Pub. No.: US 2008/0214622 A1
(43) Pub. Date: Sep. 4, 2008

(54) Substituted Triazole Derivatives As Oxytocin Antagonists

(76) Inventors: Alan Daniel Brown, Kent (GB); David Ellis, Kent (GB); Christophen Ronald Smith, San Diego, CA (US)

Correspondence Address: PFIZER INC., PATENT DEPARTMENT, MS8260-1611, EASTERN POINT ROAD, GROTON, CT, 06340, US

(21) Appl. No.: 11/909,288

(22) PCT Filed: Mar. 8, 2006
(86) PCT No.: PCT/IB2006/000676

§ 371 Date: Nov. 19, 2007

Related U.S. Application Data
(60) Provisional application No. 60/664,092, filed on Mar. 21, 2005.

(51) Int. Cl. A61K 31/4439; C07D 249/08; A61K 31/4196; C07D 401/04

(52) U.S. Cl. 514/340; 548/267.6; 514/383; 546/272.4

(57) ABSTRACT

The present invention relates to a class of substituted triazoles of formula (I) with activity as oxytocin antagonists, uses thereof, processes for the preparation thereof and compositions containing said inhibitors. These inhibitors have utility in a variety of therapeutic areas including sexual dysfunction, particularly premature ejaculation (P.E.).

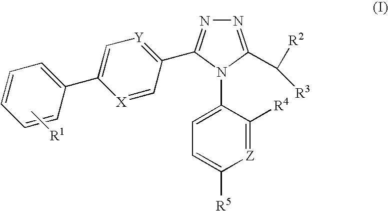

The present invention relates to a class of substituted triazoles with activity as oxytocin antagonists, uses thereof, processes for the preparation thereof and compositions containing said inhibitors. These inhibitors have utility in a variety of therapeutic areas including sexual dysfunction, particularly premature ejaculation (P.E.).

In a first aspect, the present invention provides for the use of a compound of formula (I)

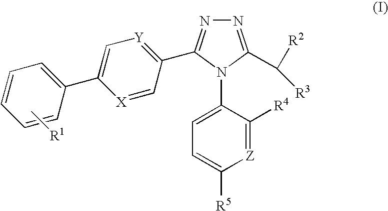

wherein:

X is C—R⁶ or N;

Y is C—R⁶ or N;

Z is C—R⁶ or N;

R¹ is selected from H, (C₁-C₆)alkyl, (C₁-C₆)alkoxy, halo, cyano, and C(O)NR⁷R⁸;

R² is selected from:
(i) H, OH, (C₁-C₆)alkoxy, OR⁹, NR⁷R⁸;
(ii) a 5-7 membered N-linked heterocycle containing 1-3 heteroatoms selected from N, O and S, optionally substituted with one or more groups selected from (C₁-C₆)alkyl, (C₁-C₆)alkoxy, oxo, and C(O)NR⁷R⁸; and
(iii) (C₁-C₆)alkyl optionally substituted with an N-linked 5-7 membered heterocycle containing 1-3 heteroatoms selected from N, O and S;

R³ is selected from H and (C₁-C₆)alkyl;

R⁴ is selected from H, (C₁-C₆)alkyl, (C₁-C₆)alkoxy, and OR⁹;

R⁵ is selected from H, halo, (C₁-C₆)alkyl, (C₁-C₆)alkoxy and NR⁷R⁸;

R⁶ is selected from H, halo, (C₁-C₆)alkyl, (C₁-C₆)alkoxy, cyano, NR⁷R⁸ and C(O)NR⁷R⁸;

R⁷ and R⁸, which may be the same or different, are selected from H, (C₁-C₆)alkyl, and C(O)R¹⁰;

R⁹ is (C₁-C₆)alkyl substituted with one or more groups selected from (C₁-C₆)alkoxy, NR⁷R⁸, and an N-linked 5-7 membered heterocycle containing 1-3 heteroatoms selected from N, O and S;

R¹⁰ is selected from (C₁-C₆)alkyl and (C₁-C₆)alkoxy;

a tautomer thereof or a pharmaceutically acceptable salt, solvate or polymorph of said compound or tautomer, in the preparation of a medicament for the treatment of sexual dysfunction.

Unless otherwise indicated, alkyl and alkoxy groups may be straight or branched and contain 1 to 6 carbon atoms and preferably 1 to 4 carbon atoms. Examples of alkyl include methyl, ethyl, n-propyl, isopropyl, n-butyl, isobutyl, sec-butyl, pentyl and hexyl. Examples of alkoxy include methoxy, ethoxy, isopropoxy and n-butoxy.

Halo means fluoro, chloro, bromo or iodo and is preferably fluoro or chloro, most preferably fluoro.

A heterocycle may be saturated, partially saturated or aromatic. Examples of heterocyclic groups are tetrahydrofuranyl, thiolanyl, pyrrolidinyl, pyrrolinyl, imidazolidinyl, imidazolinyl, sulfolanyl, dioxolanyl, dihydropyranyl, tetrahydropyranyl, piperidinyl, pyrazolinyl, pyrazolidinyl, dioxanyl, morpholinyl, dithianyl, thiomorpholinyl, piperazinyl, azepinyl, oxazepinyl, thiazepinyl, thiazolinyl and diazapanyl. Examples of aromatic heterocyclic groups are pyrrolyl, furanyl, thiophenyl, pyrazolyl, imidazolyl, isoxazolyl, oxazolyl, isothiazolyl, thiazolyl, 1,2,3-triazolyl, 1,2,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-3,4-diazolyl, tetrazolyl, pyridinyl, pyridazinyl, pyrimidinyl, pyrazinyl and triazinyl. Examples of bicyclic aromatic heterocyclic groups are benzofuranyl, benzothiophenyl, indolyl, benzimidazolyl, indazolyl, benzotriazolyl, quinolinyl and isoquinolinyl.

Unless otherwise indicated, the term substituted means substituted by one or more defined groups. In the case where groups may be selected from a number of alternative groups, the selected groups may be the same or different.

With respect to the first aspect, the following embodiments are preferred:

Preferably X is C—R⁶.

Preferably R¹ is selected from H, (C₁-C₆)alkyl, (C₁-C₆)alkoxy, cyano, and C(O)NR⁷R⁸.

Preferably R² is selected from:
(i) H, OH, and (C₁-C₆)alkoxy;
(ii) a 5-7 membered N-linked heterocycle containing 1-3 heteroatoms selected from N, O and S, optionally substituted with one or more groups selected from (C₁-C₆)alkyl, (C₁-C₆)alkoxy, oxo, and C(O)NR⁷R⁸; and
(iii) (C₁-C₆)alkyl optionally substituted with an N-linked 5-7 membered heterocycle containing 1-3 heteroatoms selected from N, O and S.

More preferably R² is selected from:
(i) H, OH, and (C₁-C₆)alkoxy; and
(ii) a 5-7 membered N-linked heterocycle containing 1-3 heteroatoms selected from N, O and S, optionally substituted with one or more groups selected from (C₁-C₆)alkyl, (C₁-C₆)alkoxy, oxo, and C(O)NR⁷R⁸.

Preferably R³ is selected from H, methyl and ethyl.

More preferably R³ is selected from H and methyl.

Preferably R⁵ is selected from halo, (C₁-C₆)alkyl, (C₁-C₆)alkoxy and NR⁷R⁸.

Preferably R⁶ is selected from H, halo, cyano, NR⁷R⁸, (C₁-C₆)alkyl, and (C₁-C₆)alkoxy,

In a second aspect, the present invention provides compounds of formula (I), as defined above, wherein:

X is C—H;

Y is C—R⁶ or N;

Z is C—H or N;

R¹ is selected from (C₁-C₆)alkyl, (C₁-C₆)alkoxy, cyano, and C(O)NR⁷R⁸;

R² is selected from:
(i) H, OH, (C₁-C₆)alkoxy; and

(ii) a 5-7 membered N-linked heterocycle containing 1-3 N atoms, optionally substituted with one or more groups selected from (C₁-C₆)alkyl, (C₁-C₆)alkoxy, oxo, and C(O)NR⁷R⁸;

R³ is H;

R⁴ is selected from H, (C₁-C₆)alkyl, (C₁-C₆)alkoxy, and OR⁹;

R⁵ is selected from halo, (C₁-C₆)alkyl, and (C₁-C₆)alkoxy;

R⁶ is selected from H, halo, (C₁-C₆)alkyl, (C₁-C₆)alkoxy, cyano, NR⁷R⁸ and C(O)NR⁷R⁸;

R⁷ and R⁸, which may be the same or different, are selected from H, (C₁-C₆)alkyl, and C(O)R¹⁰;

R⁹ is (C₁-C₆)alkyl substituted with one or more groups selected from (C₁-C₆)alkoxy, NR⁷R⁸, and an N-linked 5-7 membered heterocycle containing at least one N atom and, optionally, an additional 1-2 heteroatoms selected from N, O and S; and

R¹⁰ is selected from (C₁-C₆)alkyl and (C₁-C₆)alkoxy;

a tautomer thereof or a pharmaceutically acceptable salt, solvate or polymorph of said compound or tautomer.

With respect to the second aspect, the following embodiments are preferred:

Preferably Z is N.

Preferably R¹ is selected from (C₁-C₄)alkyl and (C₁-C₄)alkoxy.

More preferably R¹ is selected from methyl and methoxy.

Most preferably R¹ is selected from 2-methyl and 2-methoxy.

Preferably R² is selected from:
(i) H, OH, and (C₁-C₃)alkoxy; and
(ii) a 5-6 membered N-linked heterocycle containing 1-3 heteroatoms selected from N, O and S, optionally substituted with one or more groups selected from (C₁-C₆)alkyl, (C₁-C₆)alkoxy, oxo, and C(O)NR⁷R⁸.

More preferably R² is selected from:
(i) H and (C₁-C₃)alkoxy; and
(ii) a 5-6 membered N-linked heterocycle containing 1-3 N atoms, optionally substituted with one or more groups selected from (C₁-C₆)alkyl, (C₁-C₆)alkoxy, oxo, and C(O)NR⁷R⁸.

More preferably still R² is selected from:
(i) H and methoxy; and
(ii) an N-linked heterocycle selected from imidazole, 1,2,3-triazole, pyrazole and pyrrolidine, each optionally substituted with one or more groups selected from (C₁-C₃)alkyl and C(O)NR⁷R⁸.

Most preferably R² is selected from:
(i) H and methoxy; and
(ii) imidazole, 1,2,3-triazole, pyrazole, pyrrolidine, 3,5-dimethylpyrazole, 2-methylimidazole and pyrrolidine-2-carboxylic acid amide.

Preferably R⁴ is selected from H, (C₁-C₂)alkyl, (C₁-C₂)alkoxy and OR⁹.

More preferably R⁴is selected from H, methyl and OR⁹.

Most preferably R⁴is selected from H and methyl.

Preferably R⁵ is selected from (C₁-C₆)alkyl and (C₁-C₆)alkoxy.

More preferably R⁵ is selected from (C₁-C₃)alkyl and (C₁-C₃)alkoxy.

Most preferably R⁵ is selected from methyl and methoxy.

Preferably R⁶ is selected from H, (C₁-C₄)alkyl and (C₁-C₄)alkoxy.

More preferably R⁶ is selected from H and (C₁-C₃)alkoxy.

Most preferably R⁶ is selected from H and methoxy.

Preferably R⁷ and R⁸, which may be the same or different, are selected from H, methyl, ethyl and C(O)R¹⁰.

More preferably R⁷ and R⁸, which may be the same or different, are selected from H and methyl.

Most preferably R⁷ and R⁸ are H.

Preferably R⁹ is (C₁-C₄)alkyl substituted with one or more groups selected from (C₁-C₄)alkoxy, NR⁷R⁸ and an N-linked 5-6 membered heterocycle containing at least one N atom and, optionally, an additional 1-2 heteroatoms selected from N, O and S.

More preferably R⁹ is (C₁-C₃)alkyl substituted with one or more groups selected from methoxy, ethoxy, NR⁷R⁸ and an N-linked 5-6 membered heterocycle containing at least one N atom and, optionally, an additional 1-2 heteroatoms selected from N and O.

Most preferably R⁹ is ethyl substituted with one or more groups selected from methoxy, NR⁷R⁸, and an N-linked 5-6 membered heterocycle containing at least one N atom and, optionally, an additional 1-2 heteroatoms selected from N and O.

Preferably R¹⁰ is selected from (C₁-C₄)alkyl and (C₁-C₄)alkoxy.

More preferably R¹⁰ is selected from (C₁-C₄)alkyl.

Most preferably R¹⁰ is methyl

Particularly preferred compounds of the invention are:

6-methoxy-3-[3-(2′-methoxybiphenyl-4-yl)-5-methyl-4H-1,2,4-triazol-4-yl]-2-methylpyridine (Example 3);

3-(2′,3-dimethoxybiphenyl-4-yl)-4-(4-methoxyphenyl)-5-methyl-4H-1,2,4-triazole (Example 10);

2-methoxy-5-[3-(methoxymethyl)-5-(2′-methylbiphenyl-4-yl)-4H-1,2,4-triazol-4-yl]pyridine (Example 14);

2-{[5-(2′-methoxybiphenyl-4-yl)-4-(4-methylphenyl)-4H-1,2,4-triazol-3-yl]methyl}-2H-1,2,3-triazole (Example 24);

6-methoxy-3-[3-(2′-methoxybiphenyl-4-yl)-5-methyl-4H-1,2,4-triazol-4-yl]-2-methylpyridine (Example 25); and

2-methoxy-5-[3-[5-(2-methylphenyl)pyridin-2-yl]-5-(2H-1,2,3-triazol-2-ylmethyl)-4H-1,2,3-triazol-4-yl]pyridine (Example 29).

In a further embodiment, the compounds of the invention are the compounds of formula (I) and tautomers thereof and pharmaceutically acceptable salts and solvates of said compound and tautomers thereof; in particular the compounds of formula (I) and tautomers thereof and pharmaceutically acceptable salts of said compound and tautomers thereof; more particularly the compounds of formula (I) and tautomers thereof.

Pharmaceutically acceptable salts of the compounds of formulas (I) comprise the acid addition salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Hemisalts of acids may also be formed, for example, hemisulphate salts.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of formula (I) with the desired acid;
(ii) by removing an acid- or base-labile protecting group from a suitable precursor of the compound of formula (I) using the desired acid or base; or
(iii) by converting one salt of the compound of formula (I) to another by reaction with an appropriate acid or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

Included within the scope of the invention are complexes such as clathrates, drug-host inclusion complexes wherein the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288, by Haleblian (August 1975).

Hereinafter all references to compounds of formula (I) include references to salts, solvates and complexes thereof and to solvates and complexes of salts thereof.

The compounds of the invention include compounds of formula (I) as hereinbefore defined, including all polymorphs and crystal habits thereof, prodrugs and isomers thereof (including optical, geometric and tautomeric isomers) as hereinafter defined and isotopically-labeled compounds of formula (I).

As indicated, so-called ‘pro-drugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in “Pro-drugs as Novel Delivery Systems”, Vol. 14, ACS Symposium Series (T. Higuchi and W. Stella) and “Bioreversible Carriers in Drug Design”, Pergamon Press, 1987 (ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in “Design of Prodrugs” by H. Bundgaard (Elsevier, 1985).

Some examples of prodrugs in accordance with the invention include

(i) where the compound of formula (I) contains a primary or secondary amino functionality, an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references. Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include
(i) where the compound of formula (I) contains a methyl group, an hydroxymethyl derivative thereof (—CH₃→—CH₂OH):
(ii) where the compound of formula (I) contains an alkoxy group, an hydroxy derivative thereof (—OR→—OH);
(iii) where the compound of formula (I) contains a tertiary amino group, a secondary amino derivative thereof (—NR¹R²→—NHR¹ or —NHR²);
(iv) where the compound of formula (I) contains a secondary amino group, a primary derivative thereof (—NHR¹→—NH₂);
(v) where the compound of formula (I) contains a phenyl moiety, a phenol derivative thereof (-Ph→-PhOH); and
(vi) where the compound of formula (I) contains an amide group, a carboxylic acid derivative thereof (—CONH₂→COOH).

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, a keto group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains a basic moiety, an acid such as tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

The present invention includes all crystal forms of the compounds of formula (I) including racemates and racemic mixtures (conglomerates) thereof. Stereoisomeric conglomerates may be separated by conventional techniques known to those skilled in the art—see, for example, “Stereochemistry of Organic Compounds” by E. L. Eliel and S. H. Wilen (Wiley, New York, 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, 13C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Also within the scope of the invention are intermediate compounds as hereinafter defined, all salts, solvates and complexes thereof and all solvates and complexes of salts thereof as defined hereinbefore for compounds of formula (I). The invention includes all polymorphs of the aforementioned species and crystal habits thereof.

When preparing compounds of formula (I) in accordance with the invention, it is open to a person skilled in the art to routinely select the form of intermediate which provides the best combination of features for this purpose. Such features include the melting point, solubility, proccessability and yield of the intermediate form and the resulting ease with which the product may be purified on isolation.

Compounds of formula (I) may be prepared in a variety of ways. The routes below illustrate one such way of preparing these compounds; the skilled person will appreciate that other routes may be equally as practicable.

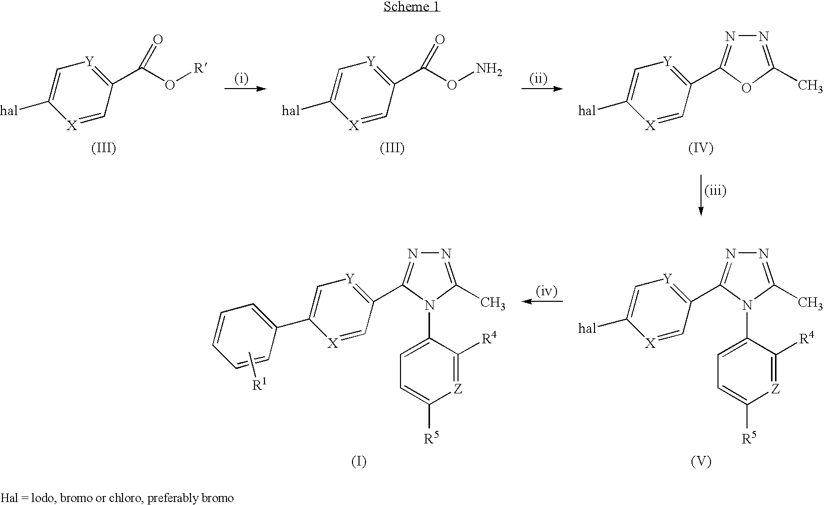

Compounds of formula (II) are either commercially available or can be prepared by analogy with the methods described in J. Org. Chem., 2001, 66, 605

Compounds of formula (III) are either commercially available or may be prepared from compounds of formula (II) by process step (i)—reaction with hydrazine monohydrate in a suitable solvent such as methanol or ethanol between −10° C. and reflux. Typical conditions comprise 1 equivalents of aryl ester (II) and 1.2-3 equivalents of hydrazine monohydrate in methanol at reflux for 18-48 hours.

Compounds of formula (IV) may be prepared from compounds of formula (III) by process step (ii)—reaction with N,N-dimethylacetamide dimethyl acetal (available from Aldrich) in a suitable solvent such as N,N-dimethylformamide, N-methyl pyrrolidine or toluene followed by the addition of a suitable acid catalyst such as trifluoroacetic acid, para-toluenesulfonic acid, camphor sulfonic acid, or hydrochloric acid. Typical conditions comprise 1 equivalent of aryl hydrazide (III) and 1.3 equivalents of N,N-dimethylacetamide dimethyl acetal in N,N-dimethylformamide heated to 60° C. for 2 hours. Concentration in vacuo, addition of toluene and 0.025 equivalents of para-toluenesulfonic acid, then heated to reflux for 2 hours.

Compounds of formula (V) may be prepared from compounds of formula (IV) by process step (iii)—reaction with a suitable aniline or 3-aminopyridine in the presence of a suitable acid, such as trifluoroacetic acid, para-toluenesulfonic acid, camphor sulfonic acid, or hydrochloric acid in a suitable solvent, such as xylene, heated at 150° C. Typical conditions comprise 1 equivalent of 1,2,4-oxadiazole (IV), 2-3 equivalents of aniline or aminopyridine and 0.04-0.1 equivalents of para-toluenesulfonic acid in xylene heated at 150° C. for 18-23 hours.

Alternatively, compounds of formula (V) may be prepared from compounds of (III) respectively by combination of process step (ii) and (iii)—sequential reaction with a dimethylacetamide dimethylacetal in a suitable solvent such as tetrahydrofuran or acetic acid heated at 55-60° C., followed by reaction with a suitable aniline or aminopyridine in the presence of a suitable acid such as acetic acid heated at 90-100° C. Typical conditions comprise 1.0 equivalent of acyl hydrazide, 1.5 equivalents of dimethylacetamide dimethylacetal (Aldrich) in tetrahydrofuran heated at 55° C. for 2 hours followed by 1.5 equivalents of aniline or aminopyridine in acetic acid heated at 90° C. for 5 hours.

Compounds of formula (I) may be prepared from compounds of formula (V) by process step (iv)—palladium mediated coupling reaction with a suitable boronic acid in a suitable solvent, such as 1,4-dioxane or dimethoxyethane/water in the presence of a suitable base such as sodium carbonate or caesium carbonate and a palladium catalyst such as [2-[(dimethylamino-κN)methyl]phenyl-κC](tricyclohexylphosphine)(trifluoro acetate-κO-(SP-4-3)-palladium, (prepared as described in Organometallics, 2003, 22, 987) or tetrakis(triphenylphosphine)palladium(0). The Palladium mediated coupling reaction can be carried out as described in the literature: Suzuki, A. Pure & Appl. Chem. 1985, 57, 1749 and reference contained within; Angew. Chem. Int. Ed. 2002, 41, 4176 and references contained within. Typical conditions comprise 1.0 equivalent of aryl halide (V), 2.5 equivalents of boronic acid, 3 equivalents caesium carbonate and 0.06 equivalents of palladium catalyst in 1,4-dioxane heated to 120° C. for 4 hours.

Compounds of general formula (I) where R³ is H and R²≠H and where R¹, R⁴, R⁵, R⁶, X, Y and Z are as described herein may be prepared according to reaction scheme 2.

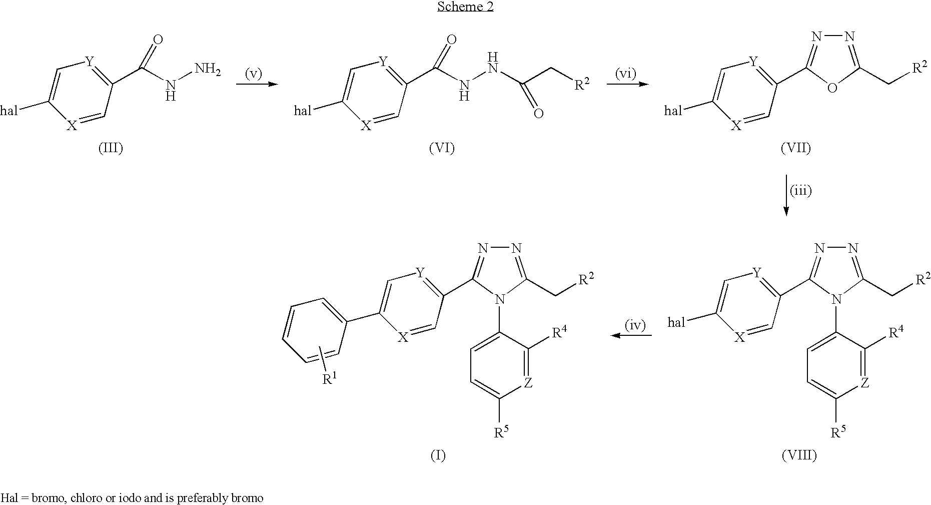

Compounds of formula (III) may be prepared as described in scheme 1.

Compounds of formula (VI) can be prepared from aryl hydrazides of formula (III) by process step (v)—reaction with an acid chloride, such as methoxyacetyl chloride, in the presence of base such as triethylamine, N-methyl morpholine, sodium carbonate or potassium hydroxide. Typical conditions comprise 1.0 equivalents of aryl hydrazide (III), 1.0-1.3 equivalents of acid chloride, 1.2-2.0 equivalents N-methyl morpholine in dichloromethane at 0-25° C. for 3-18 hours.

Compounds of formula (VII) can be prepared from compound (VI) by process step (vi)—reaction with a suitable dehydrating agent such as phosphorous oxychloride, trifluoromethanesulfonic anhydride, or phosphorous pentachloride between a temperature of 25° C. and 110° C. Typical conditions comprise 1.0 equivalents of (VI) in phosphorous oxychloride at 110° C. for 4 hours.

Compounds of formula (VIII) may be prepared from compounds of formula (VII) by process step (iii)—reaction with a suitable aniline or 3-aminopyridine in the presence of a suitable acid, such as trifluoroacetic acid, para-toluenesulfonic acid, camphor sulfonic acid, or hydrochloric acid, in a suitable solvent such as xylene heated at 150° C. Typical conditions comprise 1 equivalent of 1,2,4-oxadiazole (VII), 3 equivalents of aniline/aminopyridine and 0.04-0.1 equivalents of para-toluenesulfonic acid in xylene heated at 150° C. for 18-22 hours.

Compounds of formula (I) may be prepared from compounds of formula (VIII) by process step (iv)—palladium mediated coupling reaction as described in Scheme 1.

Compounds of general formula (I) where R² is NR⁷R⁸, R³ is H and where R¹, R⁴, R⁵, R⁶, R⁷, R⁸, X, Y and Z are as described herein may be prepared according to reaction scheme 3.

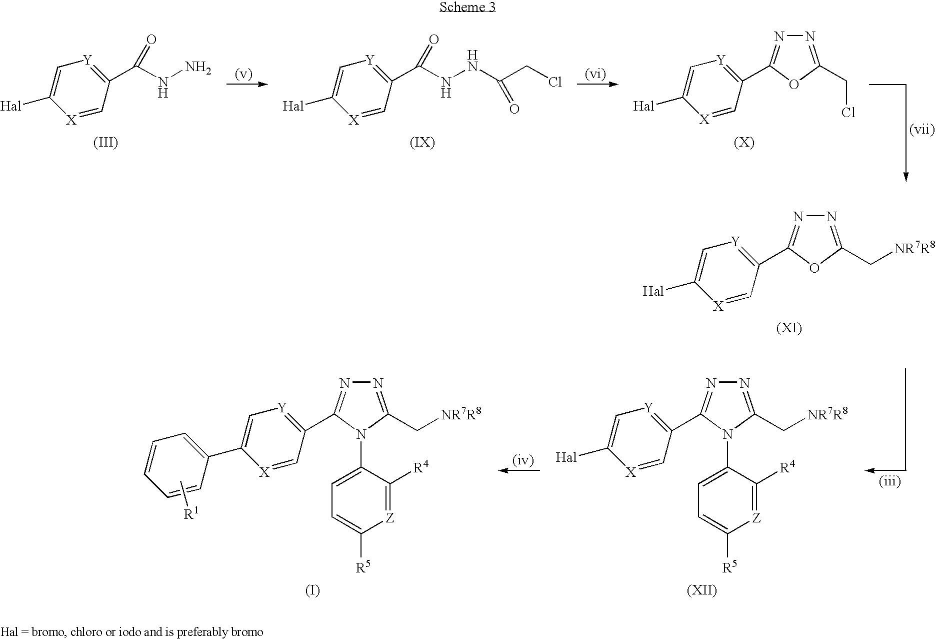

Compounds of formula (IX) can be prepared from aryl hydrazides of formula (III) by process step (v)—reaction with a suitable acid chloride, such as chloroacetyl chloride, in the presence of a base, such as triethylamine, N-methyl morpholine, sodium carbonate or potassium hydroxide. Typical conditions comprise 1.0 equivalents of aryl hydrazide (III), 1.0-1.3 equivalents of chloroacetyl chloride, 1.2-2.0 equivalents of N-methyl morpholine in dichloromethane at 25° C.

Compounds of formula (X) can be prepared from compounds of formula (IX) by process step (vi) as described in Scheme 1

Compounds of formula (XI) can be prepared from alkyl chlorides of formula (X) by process step (vii)—reaction with a suitable primary or secondary amine HNR⁷R⁸, optionally in the presence of a base such as potassium carbonate, sodium carbonate or cesium carbonate, in a suitable solvent such as acetonitrile or N,N-dimethylformamide, heating at 25° C.-50° C. for 2-18 hours. Typical conditions comprise 1 equivalent of alkyl chloride (X), 1.5 equivalent of amine (HNR⁷R⁸) and 2 equivalents of potassium carbonate in acetonitrile for 18 hours at 25° C.

Compounds of formula (XII) may be prepared from compounds of formula (XI) by process step (iii) as described in Scheme 1

Compounds of formula (I) may be prepared from compounds of formula (XII) by process step (iv) as described in Scheme 1.

Compounds of general formula I where R¹, R² R³, R⁴, R⁵, R⁶ X, Y and Z are as described herein may alternatively be prepared according to reaction scheme 4.

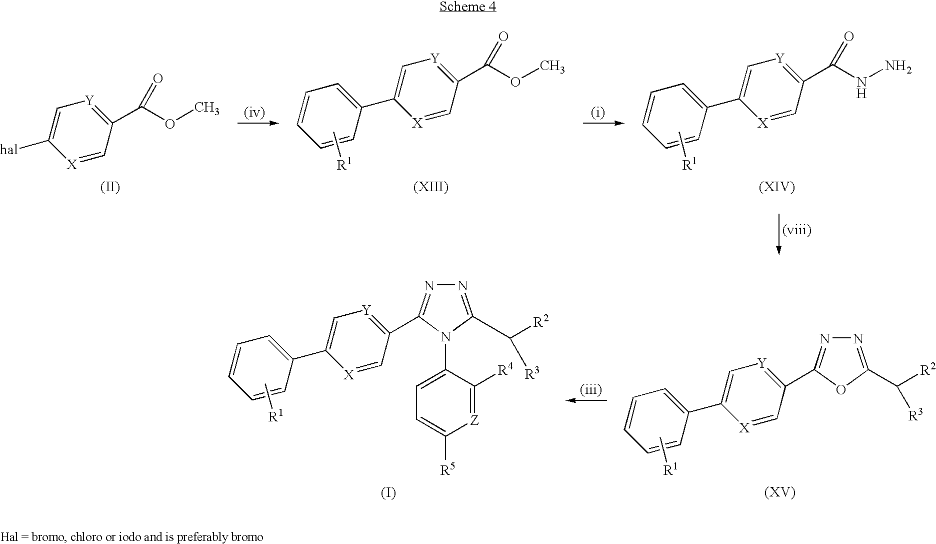

Compounds of formula (II) are either commercially available or can be prepared by analogy with the methods described in J. Org. Chem., 2001, 66, 605

Compounds of general formula (XIII) can be prepared from compounds of general formula (II) by process step (iv) as described in Scheme 1.

Compounds of general formula (XIV) can be prepared from compounds of general formula (XIII) by process step (i) as described in Scheme 1.

When R²═H, compounds of general formula (XV) can be prepared from compounds of general formula (XIV) by process step (viii), using a method analogous to process step ii, as described in scheme 1.

When R²≠H, compounds of general formula (XV) can be prepared from compounds of general formula (XIV) by process step viii, using methods analogous to steps (v) and (vi), as described in scheme 2 or steps (v), (vi) and (vii) as described in Scheme 3.

Compounds of general formula (I) can be prepared from compounds of general formula (XVI) by process step (iii), as described in Scheme 1.

Alternatively compounds of formula (I) may be prepared from compounds of formula (XIV) by combination of steps (viii) and (iii)—sequential reaction with a dimethylacetamide dimethylacetal in a suitable solvent such as tetrahydrofuran or acetic acid heated at 55-60° C. followed by reaction with a suitable aniline or aminopyridine in the presence of a suitable acid such as acetic acid heated at 90-100° C. Typical conditions comprise 1.0 equivalent of acyl hydrazide, 1.5 equivalents of dimethylacetamide dimethylacetal in tetrahydrofuran heated at 55° C. for 2 hours followed by 1.5 equivalents of 2-methoxy-5-amino-pyridine in acetic acid heated at 90° C. for 5 hours.

Alternatively, compounds of general formula (I) where X is C—R⁶, R³ is H and where R¹, R², R⁴, R⁵, R⁶ X, Y and Z are as described herein may alternatively be prepared according to reaction scheme 5.

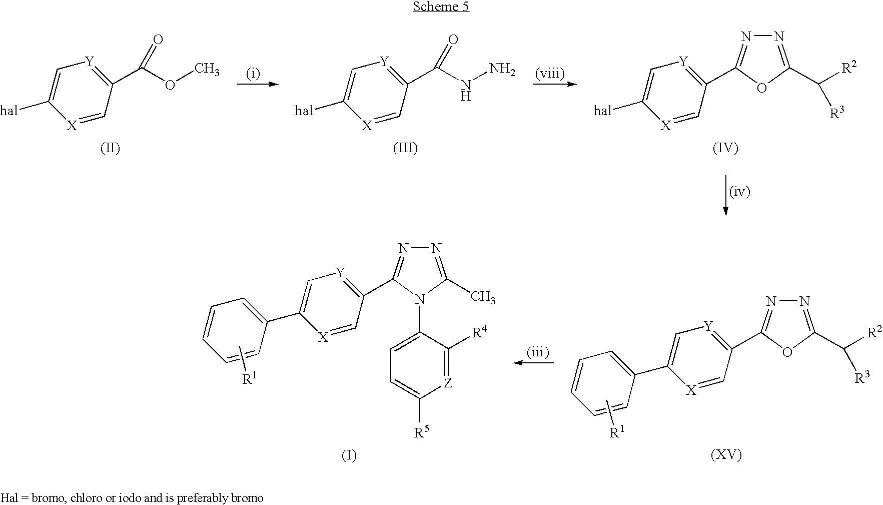

Compounds of formula (II) are either commercially available or can be prepared by analogy with the methods described in J. Org. Chem., 2001, 66, 605

Compounds of general formula (III) can be prepared from compounds of general formula (II) by process step (i) as described in Scheme 1.

When R²═H, compounds of general formula (IV) can be prepared from compounds of general formula (III) by process step (viii), using a method analogous to process step (ii), as described in scheme 1.

When R²≠H, compounds of general formula (IV) can be prepared from compounds of general formula (III) by process step (viii), using methods analogous to steps (v) and (vi), as described in Scheme 2 and steps (v), (vi) and (vii) as described in Scheme 3.

Compounds of general formula (XV) may be prepared from compounds of general formula (IV) by process step (iv) as described in Scheme 1.

Compounds of general formula (I) may be prepared from compounds of general formula (XVI) by process step (iii) as described in Scheme 1.

In a further embodiment, compounds of formula (I) may also be converted to alternative compounds of formula (I) using standard chemical reactions and transformations. For example, when R⁴≠H, a series of alkoxides are afforded by either nucleophilic substitution (as exemplified in examples 32-34) or O-alkylation (example 35) of the R⁴ group.

All of the above reactions and the preparations of novel starting materials disclosed in the preceding methods are conventional and appropriate reagents and reaction conditions for their performance or preparation as well as procedures for isolating the desired products will be well known to those skilled in the art with reference to literature precedents and the examples and preparations hereto.

The compounds of the invention are useful because they have pharmacological activity in mammals, including humans. More particularly, they are useful in the treatment or prevention of a disorder in which modulation of the levels of oxytocin could provide a beneficial effect. Disease states that may be mentioned include sexual dysfunction, particularly premature ejaculation, preterm labour, complications in labour, appetite and feeding disorders, benign prostatic hyperplasia, premature birth, dysmenorrhoea primary and secondary), congestive heart failure, arterial hypertension, liver cirrhosis, nephrotic hypertension, occular hypertension, obsessive compulsive disorder and neuropsychiatric disorders.

In another aspect the invention provides a method of treatment of a disorder or condition where inhibition of oxytocin is known, or can be shown, to produce a beneficial effect, in a mammal, comprising administering to said mammal a therapeutically effective amount of a compound of formula (I) or a pharmaceutically acceptable salt, solvate or polymorph thereof.

In another aspect the invention provides a compound of formula (I) or a pharmaceutically acceptable salt, solvate or polymorph thereof, for use in the treatment of a disorder or condition where inhibition of oxytocin is known, or can be shown, to produce a beneficial effect.

In another aspect the invention provides the use of a compound of formula (I) or a pharmaceutically acceptable salt, solvate or polymorph thereof, in the preparation of a medicament for the treatment of a disorder or condition where inhibition of oxytocin is known, or can be shown, to produce a beneficial effect, wherein the disorder or condition is selected from sexual dysfunction, male sexual dysfunction, female sexual dysfunction, hypoactive sexual desire disorder, sexual arousal disorder, orgasmic disorder, sexual pain disorder, premature ejaculation, preterm labour, complications in labour, appetite and feeding disorders, benign prostatic hyperplasia, premature birth, dysmenorrhoea, congestive heart failure, arterial hypertension, liver cirrhosis, nephrotic hypertension, occular hypertension, obsessive compulsive disorder and neuropsychiatric disorders.

In another aspect the invention provides a method of treatment of a disorder or condition where inhibition of oxytocin is known, or can be shown, to produce a beneficial effect, in a mammal, comprising administering to said mammal a therapeutically effective amount of a compound of formula (I) or a pharmaceutically acceptable salt, solvate or polymorph thereof, wherein the disorder or condition is selected from sexual dysfunction, male sexual dysfunction, female sexual dysfunction, hypoactive sexual desire disorder, sexual arousal disorder, orgasmic disorder, sexual pain disorder, premature ejaculation, preterm labour, complications in labour, appetite and feeding disorders, benign prostatic hyperplasia, premature birth, dysmenorrhoea, congestive heart failure, arterial hypertension, liver cirrhosis, nephrotic hypertension, occular hypertension, obsessive compulsive disorder and neuropsychiatric disorders.

In another aspect the invention provides a compound of formula (I) or a pharmaceutically acceptable salt, solvate or polymorph thereof, for use in the treatment of a disorder or condition where inhibition of oxytocin is known, or can be shown, to produce a beneficial effect, wherein the disorder or condition is selected from sexual dysfunction, male sexual dysfunction, female sexual dysfunction, hypoactive sexual desire disorder, sexual arousal disorder, orgasmic disorder, sexual pain disorder, premature ejaculation, preterm labour, complications in labour, appetite and feeding disorders, benign prostatic hyperplasia, premature birth, dysmenorrhoea, congestive heart failure, arterial hypertension, liver cirrhosis, nephrotic hypertension, occular hypertension, obsessive compulsive disorder and neuropsychiatric disorders.

The compounds of the invention are also useful in the treatment or prevention of anxiety, cardiovascular disease (including angina, atherosclerosis, hypertension, heart failure, edema, hypernatremia), inappropriate secretion of vasopressin, endometriosis, emesis (including motion sickness), intrauterine growth retardation, inflammation (including rheumatoid arthritis), mittelschmerz, preclampsia, premature ejaculation, premature (preterm) labor and Raynaud's disease.

Sexual dysfunction (SD) is a significant clinical problem which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes self-esteem and disrupts personal relationships thereby inducing personal distress. In the clinic, SD disorders have been divided into female sexual dysfunction (FSD) disorders and male sexual dysfunction (MSD) disorders (Melman et al, J. Urology, 1999, 161, 5-11).

FSD can be defined as the difficulty or inability of a woman to find satisfaction in sexual expression. FSD is a collective term for several diverse female sexual disorders (Leiblum, S. R. (1998). Definition and classification of female sexual disorders. Int. J. Impotence Res., 10, S104-S106; Berman, J. R., Berman, L. & Goldstein, I. (1999). Female sexual dysfunction: Incidence, pathophysiology, evaluations and treatment options. Urology, 54, 385-391). The woman may have lack of desire, difficulty with arousal or orgasm, pain with intercourse or a combination of these problems. Several types of disease, medications, injuries or psychological problems can cause FSD. Treatments in development are targeted to treat specific subtypes of FSD, predominantly desire and arousal disorders.

The categories of FSD are best defined by contrasting them to the phases of normal female sexual response: desire, arousal and orgasm (Leiblum, S. R. (1998). Definition and classification of female sexual disorders, Int. J. Impotence Res., 10, S104-S106). Desire or libido is the drive for sexual expression. Its manifestations often include sexual thoughts either when in the company of an interested partner or when exposed to other erotic stimuli. Arousal is the vascular response to sexual stimulation, an important component of which is genital engorgement and includes increased vaginal lubrication, elongation of the vagina and increased genital sensation/sensitivity. Orgasm is the release of sexual tension that has culminated during arousal.

Hence, FSD occurs when a woman has an inadequate or unsatisfactory response in any of these phases, usually desire, arousal or orgasm. FSD categories include hypoactive sexual desire disorder, sexual arousal disorder, orgasmic disorders and sexual pain disorders. Although the compounds of the invention will improve the genital response to sexual stimulation (as in female sexual arousal disorder), in doing so it may also improve the associated pain, distress and discomfort associated with intercourse and so treat other female sexual disorders.

Thus, in accordance with a further aspect of the invention, there is provided the use of a compound of the invention in the preparation of a medicament for the treatment or prophylaxis of hypoactive sexual desire disorder, sexual arousal disorder, orgasmic disorder and sexual pain disorder, more preferably for the treatment or prophylaxis of sexual arousal disorder, orgasmic disorder, and sexual pain disorder, and most preferably in the treatment or prophylaxis of sexual arousal disorder.

Hypoactive sexual desire disorder is present if a woman has no or little desire to be sexual, and has no or few sexual thoughts or fantasies. This type of FSD can be caused by low testosterone levels, due either to natural menopause or to surgical menopause. Other causes include illness, medications, fatigue, depression and anxiety.

Female sexual arousal disorder (FSAD) is characterised by inadequate genital response to sexual stimulation. The genitalia do not undergo the engorgement that characterises normal sexual arousal.

The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants eg SSRIs or antihypertensive agents.

Sexual pain disorders (includes dyspareunia and vaginismus) is characterised by pain resulting from penetration and may be caused by medications which reduce lubrication, endometriosis, pelvic inflammatory disease, inflammatory bowel disease or urinary tract problems.

The prevalence of FSD is difficult to gauge because the term covers several types of problem, some of which are difficult to measure, and because the interest in treating FSD is relatively recent. Many women's sexual problems are associated either directly with the female aging process or with chronic illnesses such as diabetes and hypertension.

Because FSD consists of several subtypes that express symptoms in separate phases of the sexual response cycle, there is not a single therapy. Current treatment of FSD focuses principally on psychological or relationship issues. Treatment of FSD is gradually evolving as more clinical and basic science studies are dedicated to the investigation of this medical problem. Female sexual complaints are not all psychological in pathophysiology, especially for those individuals who may have a component of vasculogenic dysfunction (eg FSAD) contributing to the overall female sexual complaint. There are at present no drugs licensed for the treatment of FSD. Empirical drug therapy includes oestrogen administration (topically or as hormone replacement therapy), androgens or mood-altering drugs such as buspirone or trazodone. These treatment options are often unsatisfactory due to low efficacy or unacceptable side effects.

The Diagnostic and Statistical Manual (DSM) IV of the American Psychiatric Association defines Female Sexual Arousal Disorder (FSAD) as being:
“a persistent or recurrent inability to attain or to maintain until completion of the sexual activity adequate lubrication-swelling response of sexual excitement. The disturbance must cause marked distress or interpersonal difficulty.”

The arousal response consists of vasocongestion in the pelvis, vaginal lubrication and expansion and swelling of the external genitalia. The disturbance causes marked distress and/or interpersonal difficulty.

FSAD is a highly prevalent sexual disorder affecting pre-, peri- and post menopausal (±HRT) women. It is associated with concomitant disorders such as depression, cardiovascular diseases, diabetes and UG disorders.

The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

Male sexual dysfunction (MSD) is generally associated with either erectile dysfunction, also known as male erectile dysfunction (MED) and/or ejaculatory disorders such as premature ejaculation, anorgasmia (unable to achieve orgasm) or desire disorders such as hypoactive sexual desire disorder (lack of interest in sex).

PE is a relatively common sexual dysfunction in men. It has been defined in several different ways but the most widely accepted is the Diagnostic and Statistical Manual of Mental Disorders IV one which states:
“PE is a lifelong persistent or recurrent ejaculation with minimal sexual stimulation before, upon or shortly after penetration and before the patient wishes it. The clinician must take into account factors that affect duration of the excitement phase, such as age, novelty of the sexual partner or stimulation, and frequency of sexual activity. The disturbance causes marked distress of interpersonal difficulty.”

The International Classification of Diseases 10 definition states:
“There is an inability to delay ejaculation sufficiently to enjoy lovemaking, manifest as either of the following: (1) occurrence of ejaculation before or very soon after the beginning of intercourse (if a time limit is required: before or within 15 seconds of the beginning of intercourse); (2) ejaculation occurs in the absence of sufficient erection to make intercourse possible. The problem is not the result of prolonged abstinence from sexual activity”

Other definitions which have been used include classification on the following criteria:
Related to partner's orgasm
Duration between penetration and ejaculation
Number of thrusts and capacity for voluntary control

Psychological factors may be involved in PE, with relationship problems, anxiety, depression, prior sexual failure all playing a role.

Ejaculation is dependent on the sympathetic and parasympathetic nervous systems. Efferent impulses via the sympathetic nervous system to the vas deferens and the epididymis produce smooth muscle contraction, moving sperm into the posterior urethra. Similar contractions of the seminal vesicles, prostatic glands and the bulbouretheral glands increase the volume and fluid content of semen. Expulsion of semen is mediated by efferent impulses originating from a population of lumber spinothalamic cells in the lumbosacral spinal cord (Coolen & Truitt, Science, 2002, 297, 1566) which pass via the parasympathetic nervous system and cause rhythmic contractions of the bulbocavernous, ischiocavernous and pelvic floor muscles. Cortical control of ejaculation is still under debate in humans. In the rat the medial pre-optic area and the paraventricular nucleus of the hypothalamus seem to be involved in ejaculation.

Ejaculation comprises two separate components—emission and ejaculation. Emission is the deposition of seminal fluid and sperm from the distal epididymis, vas deferens, seminal vesicles and prostrate into the prostatic urethra. Subsequent to this deposition is the forcible expulsion of the seminal contents from the urethral meatus. Ejaculation is distinct from orgasm, which is purely a cerebral event. Often the two processes are coincidental.

A pulse of oxytocin in peripheral serum accompanies ejaculation in mammals. In man oxytocin but not vasopressin plasma concentrations are significantly raised at or around ejaculation. Oxytocin does not induce ejaculation itself; this process is 100% under nervous control via α1-adrenoceptor/sympathetic nerves originating from the lumbar region of the spinal cord. The systemic pulse of oxytocin may have a role in the peripheral ejaculatory response. It could serve to modulate the contraction of ducts and glandular lobules throughout the male genital tract, thus influencing the fluid volume of different ejaculate components for example. Oxytocin released centrally into the brain could influence sexual behaviour, subjective appreciation of arousal (orgasm) and latency to subsequent ejaculation.

Accordingly, one aspect of the invention provides for the use of a compound of formula (I) in the preparation of a medicament for the prevention or treatment of sexual dysfunction, preferably male sexual dysfunction, most preferably premature ejaculation.

It has been demonstrated in the scientific literature that the number of oxytocin receptors in the uterus increases during pregnancy, most markedly before the onset of labour (Gimpl & Fahrenholz, 2001, Physiological Reviews, 81 (2), 629-683.). Without being bound by any theory it is known that the inhibition of oxytocin can assist in preventing preterm labour and in resolving complications in labour.

Accordingly, another aspect of the invention provides for the use of a compound of formula (I) in the preparation of a medicament for the prevention or treatment of preterm labour and complications in labour.

Oxytocin has a role in feeding; it reduces the desire to eat (Arletti et al., Peptides, 1989, 10, 89). By inhibiting oxytocin it is possible to increase the desire to eat. Accordingly oxytocin inhibitors are useful in treating appetite and feeding disorders.

Accordingly, a further aspect of the invention provides for the use of a compound of formula (I) in the preparation of a medicament for the prevention or treatment of appetite and feeding disorders.

Oxytocin is implicated as one of the causes of benign prostatic hyperplasia (BPH). Analysis of prostate tissue have shown that patients with BPH have increased levels of oxytocin (Nicholson & Jenkin, Adv. Exp. Med. & Biol., 1995, 395, 529). Oxytocin antagonists can help treat this condition.

Accordingly, another aspect of the invention provides for the use of a compound of formula (I) in the preparation of a medicament for the prevention or treatment of benign prostatic hyperplasia.

Oxytocin has a role in the causes of dysmenorrhoea due to its activity as a uterine vasoconstrictor (Akerlund, Ann. NY Acad. Sci., 1994, 734, 47). Oxytocin antagonists can have a therapeutic effect on this condition.

Accordingly, a further aspect of the invention provides for the use of a compound of formula (I) in the preparation of a medicament for the prevention of treatment of dysmenorrhoea.

It is to be appreciated that all references herein to treatment include curative, palliative and prophylactic treatment.

A suitable assay for determining the Oxytocin antagonist activity of a compound is detailed in International Patent Application WO 2004/020414. Using this Oxytocin Receptor Beta-lactamase assay the compounds of the present invention all exhibit Ki values of less than 250 nM. The compound of Example 2 has a Ki value of 11.2 nM. The compound of Example 10 has a Ki value of 14.6 nM. The compound of Example 25 has a Ki value of 11.7 nM. The compound of Example 32 has a Ki value of 13.8 nM.

The compounds of the present invention may be coadministered with one or more agents selected from:
1) One or more selective serotonin reuptake inhibitors (SSRIs) such as dapoxetine, paroxetine, 3-[(dimethylamino)methyl]-4-[4-(methylsulfanyl)phenoxy]benzenesulfonamide (Example 28, WO 0172687), 3-[(dimethylamino)methyl]-4-[3-methyl-4-(methylsulfanyl)phenoxy]benzenesulfonamide (Example 12, WO 0218333), N-methyl-N-({3-[3-methyl-4-(methylsulfanyl)phenoxy]-4-pyridinyl}methyl)amine (Example 38, PCT Application no PCT/IB02/01032).
2) One or more local anaesthetics;
3) one or more α-adrenergic receptor antagonists (also known as α-adrenoceptor blockers, α-receptor blockers or α-blockers); suitable α₁-adrenergic receptor antagonists include: phentolamine, prazosin, phentolamine mesylate, trazodone, alfuzosin, indoramin, naftopidil, tamsulosin, phenoxybenzamine, rauwolfa alkaloids, Recordati 15/2739, SNAP 1069, SNAP 5089, RS17053, SL 89.0591, doxazosin, Example 19 of WO9830560, terazosin and abanoquil; suitable α₂- adrenergic receptor antagonists include dibenarnine, tolazoline, trimazosin, efaroxan, yohimbine, idazoxan clonidine and dibenarnine; suitable non-selective α-adrenergic receptor antagonists include dapiprazole; further α-adrenergic receptor antagonists are described in PCT application WO99/30697 published on 14 Jun. 1998 and U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference;
4) one or more cholesterol lowering agents such as statins (e.g. atorvastatin/Lipitor—trade mark) and fibrates;
5) one or more of a serotonin receptor agonist, antagonist or modulator, more particularly agonists, antagonists or modulators for example 5HT1A, 5HT2A, 5HT2C, 5HT3, 5HT6 and/or 5HT7 receptors, including those described in WO-09902159, WO-00002550 and/or WO-00028993;
6) one or more NEP inhibitors, preferably wherein said NEP is EC 3.4.24.11 and more preferably wherein said NEP inhibitor is a selective inhibitor for EC 3.4.24.11, more preferably a selective NEP inhibitor is a selective inhibitor for EC 3.4.24.11, which has an IC₅₀ of less than 100 nM (e.g. ompatrilat, sampatrilat) suitable NEP inhibitor compounds are described in EP-A-1097719; IC50 values against NEP and ACE may be determined using methods described in published patent application EP1097719-A1, paragraphs [0368] to [0376];
7) one or more of an antagonist or modulator for vasopressin receptors, such as relcovaptan (SR 49059), conivaptan, atosiban, VPA-985, CL-385004, Vasotocin.
8) Apomorphine—teachings on the use of apomorphine as a pharmaceutical may be found in U.S. Pat. No. 5,945,117;
9) Dopamine agonists (in particular selective D2, selective D3, selective D4 and selective D2-like agents) such as Pramipexole (Pharmacia Upjohn compound number PNU95666), ropinirole, apomorphine, surmanirole, quinelorane, PNU-142774, bromocriptine, carbergoline, Lisuride, R-(−)-3-(4-Propylmorpholin-2-yl)phenol or 5-[(2R,5S)-5-methyl-4-propylmorpholin-2-yl]pyridin-2-amine, and 2-[(4-pyridin-2-ylpiperazin-1-yl)methyl]-1H-benzimidazole (ABT724);
10) Melanocortin receptor agonists (e.g. Melanotan II and PT141) and selective MC3 and MC4 agonists (e.g. THIQ);
11) Mono amine transport inhibitors, particularly Noradrenaline Re-uptake Inhibitors (NRIs) (e.g. Reboxetine), other Serotonin Re-uptake Inhibitors (SRIs) (e.g. paroxetine, dapoxetine) or Dopamine Re-uptake Inhibitors (DRIs);
12) 5-HT_{1A} antagonists (e.g. robalzotan); and
13) PDE inhibitors such as PDE2 (e.g. erythro-9-(2-hydroxyl-3-nonyl)-adenine) and Example 100 of EP 0771799—incorporated herein by reference) and in particular a PDE5 inhibitor such as the pyrazolo[4,3-d]pyrimidin-7-ones disclosed in EP-A-0463756; the pyrazolo[4,3-d]pyrimidin-7-ones disclosed in EP-A-0526004; the pyrazolo[4,3-d]pyrimidin-7-ones disclosed in published international patent application WO 93/06104; the isomeric pyrazolo[3,4-d]pyrimidin-4-ones disclosed in published international patent application WO 93/07149; the quinazolin-4-ones disclosed in published international patent application WO 93/12095; the pyrido[3,2-d]pyrimidin-4-ones disclosed in published international patent application WO 94/05661; the purin-6-ones disclosed in published international patent application WO 94/00453; the pyrazolo[4,3-d]pyrimidin-7-ones disclosed in published international patent application WO 98/49166; the pyrazolo[4,3-d]pyrimidin-7-ones disclosed in published international patent application WO 99/54333; the pyrazolo[4,3-d]pyrimidin-4-ones disclosed in EP-A-0995751; the pyrazolo[4,3-d]pyrimidin-7-ones disclosed in published international patent application WO 00/24745; the pyrazolo[4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124. The pyrazolo[4,3-d]pyrimidin-7-ones disclosed in published international application WO 01/27112; the pyrazolo[4,3-d]pyrimidin-7-ones disclosed in published international application WO 01/27113; the compounds disclosed in EP-A-1092718 and the compounds disclosed in EP-A-1092719.
Preferred PDE5 inhibitors for use with the invention:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);
5-(2-ethoxy-5-morpholinoacetylphenyl)-1-methyl-3-n-propyl-1,6-dihydro-7H-pyrazolo[4,3-d]pyrimidin-7-one (see EP-A-0526004);
3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);
3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333);
(+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333);
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);
5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);
5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);
5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124);
5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);
(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8;
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and
the compound of example 11 of published international application WO93/07124 (EISAI); and compounds 3 and 14 from Rotella D P, J. Med. Chem., 2000, 43, 1257.
Still further PDE5 inhibitors for use with the invention include:
4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)arnino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

The contents of the published patent applications and journal articles and in particular the general formulae of the therapeutically active compounds of the claims and exemplified compounds therein are incorporated herein in their entirety by reference thereto.

More preferred PDE5 inhibitors for use with the invention are selected from the group:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil);
(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351);
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil); and
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one or 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one and pharmaceutically acceptable salts thereof.

A particularly preferred PDE5 inhibitor is 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) (also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine) and pharmaceutically acceptable salts thereof. Sildenafil citrate is a preferred salt.

Preferred agents for coadministration with the compounds of the present invention are PDE5 inhibitors, selective serotonin reuptake inhibitors (SSRIs), vasopressin V_{1A} antagonists, α-adrenergic receptor antagonists, NEP inhibitors, dopamine agonists and melanocortin receptor agonists as described above. Particularly preferred agents for coadministration are PDE5 inhibitors, SSRIs, and V_{1A} antagonists as described herein.

The compounds of the formula (I) can be administered alone but will generally be administered in admixture with a suitable pharmaceutical excipient, diluent or carrier selected with regard to the intended route of administration and standard pharmaceutical practice.

The present invention provides for a composition comprising a compound of formula (I) and a pharmaceutically acceptable diluent or carrier.

Compounds of the invention intended for pharmaceutical use may be administered as crystalline or amorphous products or may exist in a continuum of solid states ranging from fully amorphous to fully crystalline. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in “Pharmaceutical Dosage Forms: Tablets”, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in “Pharmaceutical Technology On-line”, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999). Other means of topical administration include delivery by electroporation, iontophoresis, phonophoresis, sonophoresis and microneedle or needle-free (e.g. Powderject™, Bioject™, etc.) injection. Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 2 to 30 mg of the compound of formula (I). The overall daily dose will typically be in the range 50 to 100 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis. Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions. Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like. The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range 50 mg to 100 mg depending, of course, on the mode of administration and efficacy. For example, oral administration may require a total daily dose of from 50 mg to 100 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

The invention is illustrated by the following non-limiting examples in which the following abbreviations and definitions are used:

Arbocel® Filtration agent, from J. Rettenmaier & Sohne, Germany

APCI+ Atmospheric Pressure Chemical Ionisation (positive scan)

CDCl₃ Chloroform-d1

d Doublet

dd Doublet of doublets

DMSO Dimethylsulfoxide

ES+ Electrospray ionisation positive scan.

eq Equivalent

¹H NMR Proton Nuclear Magnetic Resonance Spectroscopy

HPLC High Performance Liquid Chromatography

HRMS High Resolution Mass Spectroscopy

LRMS Low Resolution Mass Spectroscopy

m Multiplet

m/z Mass spectrum peak

q Quartet

s Singlet

t Triplet

δ Chemical shift

### EXAMPLE 1

### 5-[3-(2′-Methoxybiphenyl-4-yl)-5-methyl-4H-1,2,4-triazol-4]-N-methylpyridin-2-amine

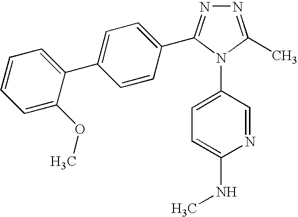

A mixture of the para-toluenesulfonic acid (4 mg) and the products of preparations 48 (300 mg, 1.1 mmol) and 58 (300 mg, 2.5 mmol) in xylene (10 mL) was heated under reflux for 18 hours. The reaction mixture was then cooled to room temperature and diluted with dichloromethane (20 mL). Polymer-supported isocyanate was added and the reaction mixture was stirred for 2 hours at room temperature, filtered and concentrated in vacuo. The residue was taken up in methanol, charcoal was added and the reaction mixture was filtered through Celite®. The residue was concentrated in vacuo and the residue was purified by column chromatography on silica gel, eluting with dichloromethane:methanol, 100:0 to 96:4, to afford the title compound as a white solid in 22% yield, 88 mg

¹HNMR(CDCl₃, 400 MHz) δ: 2.39(s, 3H), 3.02-3.06(m, 3H), 3.80(s, 3H), 5.70(bs, 1H), 6.56(d, 1H), 6.97(d, 1H), 7.01 (m, 1H), 7.24-7.36(m, 3H), 7.51 (m, 4H), 7.98(d, 1H); LRMS APCI m/z 372 [M+H]⁺

### EXAMPLE 2

### 4-[2-({6-Methoxy-3-[3-(2′-methoxybiphenyl-4-yl)-5-methyl-4H-1,2,4-triazol-4-yl]pyridin-2-yl}oxy)ethyl]morpholine

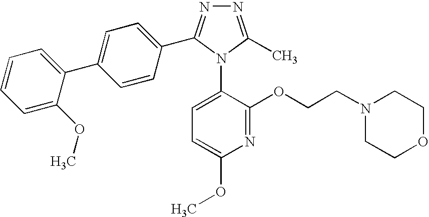

The title compound was prepared from the products of preparations 48 and 60, using the same method as example 1. The crude compound was treated with charcoal and re-purified by column chromatography on silica gel, eluting with dichloromethane:methanol, 99.5:0.5, to afford the title compound in 2% yield.

¹HNMR(400 MHz, CDCl₃) δ: 2.32(s, 3H), 2.33-2.68(m, 6H), 3.59-3.78(m, 4H), 3.80(s, 3H), 3.97(s, 3H), 4.47-4.53(m, 2H), 6.44(d, 1H), 6.96(d, 1H), 7.00(t, 1H), 7.27(d, 1H), 7.31(t, 1H), 7.39(d, 1H), 7.48(d, 2H), 7.51 (d, 2H); LRMS ESI m/z 524 [M+Na]⁺

### EXAMPLE 3

### 6-Methoxy-3-[3-(2′-methoxybiphenyl-4-yl)-5-methyl-4H-1,2,4-triazol-4-yl]-2-methylpyridine

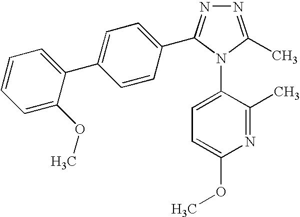

A mixture of the product of preparation 48 (100 mg, 0.38 mmol), 5-amino-2-methoxypyridine [(78 mg, 0.56 mmol), WO 2004062665, p 46] and para-toluenesulfonic acid (cat) in xylene (10 mL) was heated under reflux for 18 hours. The reaction mixture was then concentrated in vacuo and the residue was re-dissolved in xylene (10 mL). 5-Amino-2-methoxypyridine (78 mg, 0.56 mmol) and para-toluenesulfonic acid (cat) were added and the mixture was heated under reflux for 18 hours. The reaction mixture was concentrated in vacuo and the residue was dissolved in ethyl acetate, washed with water, citric acid, sodium hydrogen carbonate solution and brine. The organic solution was then dried over sodium sulfate, concentrated in vacuo and the residue was purified by column chromatography on silica gel, eluting with dichloromethane:methanol, 100:0 to 98:2, to afford the title compound in 36% yield, 52 mg

¹HNMR(CDCl₃, 400 MHz) δ: 2.20(s, 3H), 2.30(s, 3H), 3.80(s, 3H), 4.00(s, 3H), 6.80(d, 1H), 7.00(m, 2H), 7.30(m, 2H), 7.40(d, 1H), 7.50(m, 4H); LRMS ESI m/z 357 [M+H]⁺

### EXAMPLE 4

### 2-Methoxy-5-{3-(methoxymethyl)-5-[6-(2-methylphenyl)pyridin-3-yl]-4H-1,2,4-triazol-4-yl}pyridine

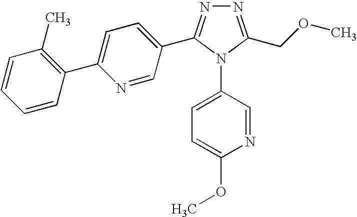

A mixture of the product of preparation 49 (758 mg, 2.22 mmol), 5-methoxypyridin-2-amine (303 mg, 2.44 mmol) and para-toluenesulfonic acid (20 mg) in xylene (10 mL) was heated at 145° C. for 18 hours. The reaction mixture was then cooled to room temperature and concentrated in vacuo. The residue was partitioned between ethyl acetate and sodium hydrogen carbonate solution, and the organic layer was separated, dried over sodium sulfate and concentrated in vacuo. The residue was then purified by column chromatography on silica gel, eluting with dichloromethane:methanol:0.88 ammonia,100:0:0 to 98:2:0.2. The resulting residue was triturated with diethyl ether to afford the title compound as a pale brown solid in 63% yield, 538 mg.

¹HNMR(400 MHz, CDCl₃) δ: 2.35(s, 3H), 3.37(s, 3H), 3.99(s, 3H), 4.51(s, 2H), 6.88(d, 1H), 7.24-7.33 (m, 3H), 7.38(d, 1H), 7.44(d, 1H), 7.55(dd, 1H), 8.00(dd, 1H), 8.16(d, 1H), 8.66(d, 1H); LRMS ESI m/z 410 [M+Na]⁺

### EXAMPLES 5 TO 8

The following compounds, of the general formula shown below, were prepared using the same method to that described for example 4, using the products of preparations 48 and 50 with commercially available amines.

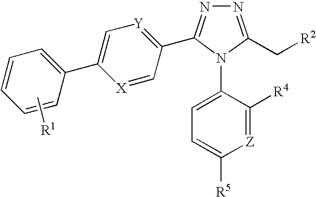

| | | |
|---|---|---|
| No. | Data | Yield |

X = CH, Y = CH

| | | |
|---|---|---|
| 5 | R¹ = 6-OCH₃, Z = CH, R² = H, R⁴ = H, R⁵ = CH³ | 48% |
| | ¹HNMR(400 MHz, CDCl₃) δ: 2.39(s, 3H), 2.46(s, 3H), | |
| | 3.78(s, 3H), 6.95(d, 1H), 6.98(d, 2H), 7.12(d, 2H), | |
| | 7.24-7.34(m, 4H), 7.44(d, 2H), 7.47(d, 2H); | |
| | LRMS APCl m/z 356 [M + H]⁺ | |
| 6 | R¹ = 6-OCH₃, Z = CH, R² = H, R⁴ = H, R⁵ = OCH₃ | 50% |
| | ¹HNMR(400 MHz, CDCl₃) δ: 2.38(s, 3H), | |
| | 3.78(s, 3H), 3.88(s, 3H), 6.93-7.05(m, 4H), 7.17(d, 2H), | |
| | 7.25-7.33(m, 2H), 7.45(d, 2H), 7.48(d, 2H); LRMS | |
| | APCl m/z 372 [M + H]⁺ | |
| 7 | R¹ = 6-OCH₃, Z = N, R² = H, R⁴ = H, R⁵ = OCH₃ | 30% |
| | ¹HNMR(400 MHz, CDCl₃) δ: 2.37(s, 3H), 3.78(s, 3H), | |
| | 4.00(s, 3H), 6.86(d, 1H), 6.95(d, 1H), 6.99(m, 1H), | |
| | 7.26(dd, 1H), 7.29(m, 1H), 7.40-7.50(m, 5H), | |
| | 8.09(d, 1H); LRMS APCl m/z 373 [M + H]⁺ | |

X = CH, Y = N

| | | |
|---|---|---|
| 8 | R¹ = 6-OCH₃, Z = CH, R² = H, R⁴ = H, R⁵ = OCH₃ | 54% |
| | ¹HNMR(400 MHz, CDCl₃) δ: 2.34(s, 3H), | |
| | 3.79(s, 3H), 3.85(s, 3H), 6.90-6.98(m, 3H), 7.01(m, 1H), | |
| | 7.16(m, 2H), 7.24-7.29(m, 1H), 7.34(m, 1H), | |
| | 7.91(m, 1H), 8.05(m, 1H), 8.48(m, 1H); | |
| | LRMS APCl m/z 395 [M + Na]⁺ | |

### EXAMPLE 9

### 2,6-dimethoxy-3-[3-(2′-methoxybiphenyl-4-yl)-5-methyl-4H-1,2,4-triazol-4-yl]pyridine

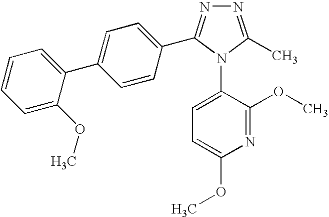

3-Amino-2,6-dimethoxypyridine monohydrochloride (130 mg, 0.67 mmol) was partitioned between ethyl acetate and sodium carbonate solution. The organic layer was separated, washed with brine, dried over sodium sulfate and concentrated in vacuo. The residue was taken up in xylene (3 mL), the product of preparation 48 (150 mg, 0.56 mmol) and para-toluenesulfonic acid (20 mg) were added and the mixture was heated under reflux for 18 hours. The reaction mixture was then concentrated in vacuo and the residue was taken up in 2M hydrochloric acid and washed with ethyl acetate. The aqueous solution was basified with sodium hydroxide and extracted with ethyl acetate. The organic solution was then washed with brine, dried over sodium sulfate and concentrated in vacuo. The residue was purified by column chromatography on silica gel, eluting with dichloromethane:methanol:methanol:0.88 ammonia, 100:0:0 to 99:1:0.1. The appropriate fractions were evaporated under reduced pressure and the residue was triturated with diethyl ether to afford the title compound as a white solid in 17% yield, 38 mg.

¹HNMR(400 MHz, CDCl₃) δ: 2.37(s, 3H), 3.80(s, 3H), 3.92(s, 3H), 4.00(s, 3H), 6.42(d, 1H), 6.96-7.03(m, 2H), 7.27-7.37(m, 3H), 7.47-7.52(m, 4H); LRMS ESI m/z 403 [M+H]⁺

### EXAMPLE 10

### 3-(2′,3-Dimethoxybiphenyl-4-yl)-4-(4-methoxyphenyl)-5-methyl-4H-1,2,4-triazole

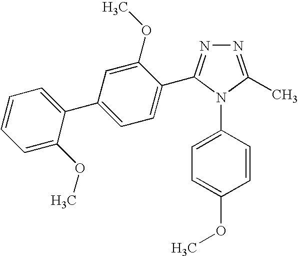

A mixture of the product of preparation 44 (160 mg, 0.43 mmol), (2-methoxyphenyl)boronic acid (208 mg, 1.38 mmol), caesium carbonate (417 mg, 1.29 mmol) and the palladium complex of preparation 47 (10 mg) in dioxane (3 mL) was heated under reflux for 4 hours. Further palladium catalyst (10 mg) (preparation 47) was added and heating continued for 2 hours. The reaction mixture was then filtered through Celite®, washing through with dichloromethane and sodium hydrogen carbonate solution. The filtrate layers were separated and the organic solution was washed with brine, dried over sodium sulfate and concentrated in vacuo. The residue was purified by column chromatography on silica gel, eluting with ethyl acetate, followed by dichloromethane:methanol:0.88 ammonia, 98:2:0.2. The resulting residue was triturated with diethyl ether to afford the title compound as a pale pink solid in 76% yield, 130 mg.

¹HNMR(400 MHz, CDCl₃) δ: 2.48, 2.58(2xs, 3H), 3.42, 3.85(2×s, 3H), 3.77,3.80(2×s, 3H), 3.83, 3.84(s, 3H), 6.91-7.04(m, 5H), 7.08-7.12(d, 2H), 7.18-7.20(dd, 1H), 7.27-7.37(m, 2H), 7.52(d, 1H); LRMS APCI m/z 825 [2M+Na]⁺

### EXAMPLE 11

### 3-(3-Methoxy-2′-methylbiphenyl-4-yl)-4-(4-methoxyphenyl)-5-methyl-4H-1,2,4-triazole

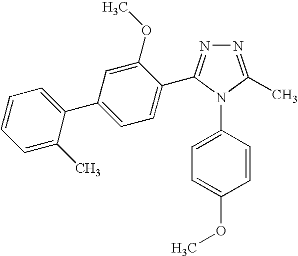

The title compound was prepared from the product of preparation 44 and (2-methylphenyl)boronic acid, using the using the same method as that described for example 10, in 60% yield.

¹HNMR(400 MHz, CDCl₃) δ: 2.23(s, 3H), 2.50(s, 3H), 3.47(s, 3H), 3.83(s, 3H), 6.67(s, 1H), 6.90(m, 3H), 6.95(m, 2H), 7.16-7.25(m, 4H), 7.52(d, 1H); LRMS APCI m/z 386 [M+H]⁺

### EXAMPLE 12

### 4′-[4-(4-Methoxyphenyl)-5-methyl-4H-1,2,4-triazol-3-yl]biphenyl-2-carboxamide

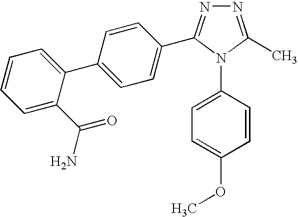

The title compound was prepared from the product of preparation 22 and [2-(aminocarbonyl)phenyl]boronic acid, using the same method as that described for example 10. The crude compound was purified by HPLC using a Phenomenex Luna C18 system, eluting with water/diethylamine (99.9:0.1):acetonitrile, 90:10 to 5:95 to afford the desired product in 3% yield.

HRMS m/z found 385.1657; C₂₃H₂₀N₄O₂ requires 385.1659 [M+H]⁺

### EXAMPLE 13

### 2-Methoxy-5-[3-(3′-methoxybiphenyl-4-yl)-5-(methoxymethyl)-4H-1,2,4-triazol-4-yl]pyridine

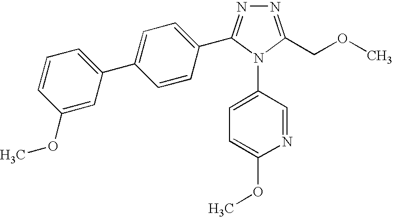

The title compound was prepared from the product of preparation 21 and (3-methoxyphenyl)boronic acid, using the same method as that described for preparation 48, as a peach solid in 61% yield.

¹HNMR(400 MHz, CDCl₃) δ: 3.38(s, 3H), 3.86(s, 3H), 4.01(s, 3H), 4.56(s, 2H), 6.85(d, 1H), 6.90(d, 2H), 7.08(m, 7H), 7.14(d, 1H), 7.34(m, 1H), 7.49-7.57(m, 5H), 8.14(d, 1H); LRMS APCI m/z 403 [M+H]⁺

### EXAMPLE 14

### 2-Methoxy-5-[3-(methoxymethyl)-5-(2′-methylbiphenyl-4-yl)-4H-1,2,4-triazol-4-yl]pyridine

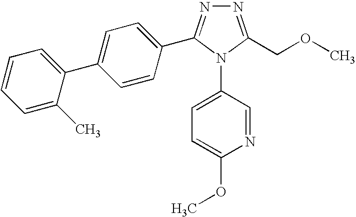

The title compound was prepared from the product of preparation 21 and (2-methylphenyl)boronic acid, using the same method as that described for preparation 48. The crude compound was purified by column chromatography on silica gel, eluting with dichloromethane:methanol:0.88 ammonia, 99:1:0.1 to 98:2:0.2. The resulting residue was triturated with diethyl ether to afford the desired product as a white solid in 60% yield.

¹HNMR(400 MHz, CDCl₃) δ: 2.24(s, 3H), 3.38(s, 3H), 4.00(s, 3H), 4.50(s, 2H), 6.87(d, 1H), 7.17-7.26(m, 3H), 7.27-7.31 (m, 3H), 7.49-7.56(m, 3H), 8.16(d, 1H); LRMS APCI m/z 387 [M+H]⁺

### EXAMPLE 15

### 2-Methoxy-5-[3-(2′-methoxybiphenyl-4-yl)-5-(methoxymethyl)-4H-1,2,4-triazol-4-yl]pyridine

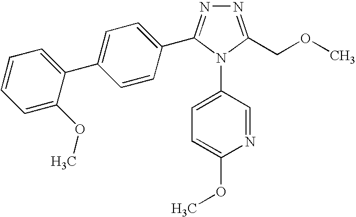

The title compound was prepared from the product of preparation 21 and (2-methoxyphenyl)boronic acid, using the same method as that described for example 14, as a white solid in 70% yield.

¹HNMR(400 MHz, CDCl₃) δ: 3.37(s, 3H), 3.80(s, 3H), 4.00(s, 3H), 4.49(s, 2H), 6.85(d, 1H), 6.96-7.04(m, 2H), 7.26-7.35(m, 2H), 7.48-7.55(m, 5H), 8.16(d, 1H); LRMS APCI m/z 403 [M+H]⁺

### EXAMPLE 16

### 4′-[4-(4-Methoxyphenyl)-5-methyl-4H-1,2,4-triazol-3-yl]biphenyl-2-carbonitrile

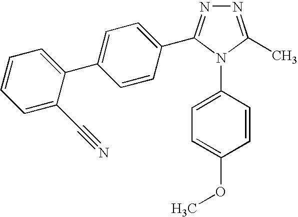

A mixture of the product of preparation 22 (100 mg, 0.29 mmol), 2-cyanophenylboronic acid (1.0 mg, 0.68 mmol), caesium carbonate (440 mg, 1.35 mmol) and the palladium complex of preparation 47 (10 mg) in dioxane (5 mL) was heated under reflux for 3 hours. The reaction mixture was filtered though a phase separation tube, washing through with dichloromethane and the organic solution was concentrated in vacuo The residue was purified by HPLC using a Phenomenex Luna C18 system, eluting with water/diethylamine (99.9:0.1):acetonitrile, 90:10 to 5:95 to afford the desired product in 73% yield, 78 mg.

HRMS m/z found 367.1554; C₂₃H₁₈N₄O requires 367.1552 [M+H]⁺

### EXAMPLE 17

### 4-(4-Methoxyphenyl)-3-methyl-5-(2′-methylbiphenyl-4-yl)-4H-1,2,4-triazole

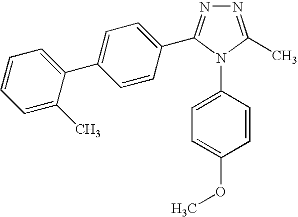

The title compound was prepared from the product of preparation 22 and 2-methylboronic acid, using the same method as that described for example 16, in 9% yield.

HRMS m/z found 356.1745; C₂₃H₂₁N₃O requires 356.1758 [M+H]⁺

### EXAMPLE 18

### 3-(1H-Imidazol-1-ylmethyl)-5-(2′-methoxybiphenyl-4-yl)-4-(4-methoxyphenyl)-4H-1,2,4-triazole

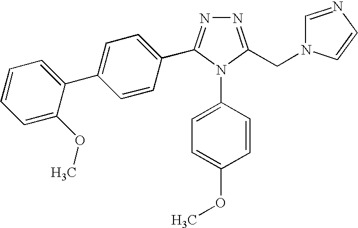

A solution of sodium carbonate (81 mg, 0.78 mmol) in water (2 mL) was added to a mixture of the product of preparation 23 (179 mg, 0.39 mmol), (2-methoxyphenyl)boronic acid (89 mg, 0.59 mmol) and tetrakis(triphenylphosphine)palladium(0) (23 mg) in 1,2-dimethoxyethane (4 mL) and the mixture was heated under reflux for 90 minutes. The reaction mixture was then concentrated in vacuo and the residue was dissolved in ethyl acetate, washed with water, sodium hydroxide solution and brine, dried over sodium sulfate and concentrated in vacuo. Purification of the residue by column chromatography on silica gel, eluting with dichloromethane:methanol, 99:1 to 97:3 afforded the title compound in 76% yield, 130 mg.

¹HNMR(400 MHz, CDCl₃) δ: 3.80(s, 3H), 3.90(s, 3H), 5.30(s, 2H), 7.00(m, 6H), 7.10(s, 1H), 7.30(m, 4H), 7.50(m, 4H); LRMS APCI m/z 438 [M+H]⁺

### EXAMPLE 19-31

The following compounds, of the general formula shown below, were prepared using the same method to that described for example 18, using the products of preparations 24-36 with either (2-methoxyphenyl)boronic acid or (2-methylphenyl)boronic acid.

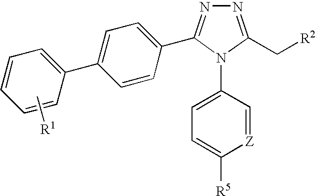

| | | | | |
|---|---|---|---|---|
| No | R² | R5 | Data | Yield |

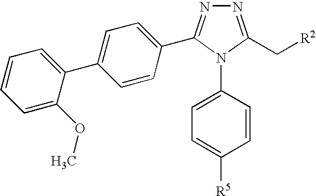

| | | | | |
|---|---|---|---|---|
| 19 | 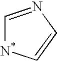 | CH₃ | ¹HNMR (400 MHz, CDCl₃) δ: 2.25 (s, 3 H), 3.80 (s,3 H), 5.20 (s, 2 H), 6.82-6.90 (m, 3 H), 6.92-7.02 (m,3 H), 7.06 (s, 1 H), 7.22-7.34 (m, 4 H), 7.46 (m, 4 H);LRMS APCl m/z 422 [M + H]⁺ | 71% |
| 20 | 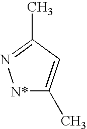 | OCH₃ | ¹HNMR (400 MHz, CDCl₃) δ: 2.10 (s, 3 H), 2.20 (s,3 H), 3.80 (s, 3 H), 3.90 (s, 3 H), 5.30 (s, 2 H),5.70 (s, 1 H), 7.00 (m, 6 H), 7.30 (m, 1 H), 7.50 (m,5 H); LRMS APCl m/z 466 [M + H]⁺ | 26% |
| 21 | 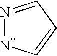 | OCH₃ | ¹HNMR (400 MHz, CDCl₃) δ: 3.80 (s, 3 H), 3.90 (s,3 H), 5.40 (s, 2 H), 6.30 (d, 1 H), 6.90-7.50 (m,14 H); LRMS APCl m/z 438 [M + H]⁺ | 38% |
| 22 | 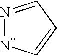 | CH₃ | ¹HNMR (400 MHz, CDCl₃) δ: 2.40 (s, 3 H), 3.80 (s,3 H), 5.40 (s, 2 H), 6.20 (d, 1 H), 6.80-7.50 (m,14 H); LRMS APCl m/z 422 [M + H]⁺ | 66% |
| 23 | 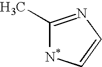 | CH₃ | ¹HNMR (400 MHz, CDCl₃) δ: 2.00 (s, 3 H), 3.80 (s,3 H), 3.90 (s, 3 H), 5.10 (s, 2 H), 6.60 (s, 1 H), 6.85-6.90(m, 3 H), 6.94-7.02 (m, 4 H), 7.26 (m, 2 H),7.47 (m, 4 H); LRMS APCl m/z 452 [M + H]⁺ | 53% |
| 24 | 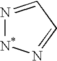 | CH₃ | ¹HNMR (400 MHz, DMSO-d₆) δ: 2.90 (s, 3 H),4.70 (s, 3 H), 5.70 (s, 2 H), 7.00 (m, 1 H), 7.10 (d,1 H), 7.20 (d, 2 H), 7.25 (m, 3 H), 7.30 (m, 3 H),7.40 (d, 2 H), 7.70 (s, 2 H); LRMS APCl m/z 423[M + H]⁺ | 77% |
| 25 | 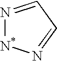 | OCH₃ | ¹HNMR (400 MHz, DMSO-d₆) δ: 3.73 (s, 3 H),3.77 (s, 3 H), 5.73 (s, 2 H), 6.97-7.00 (m, 3 H),7.10 (d, 1 H), 7.18 (d, 2 H), 7.22-7.29 (m, 1 H),7.31-7.37 (m, 1 H), 7.40 (m, 2 H), 7.44 (m, 2 H),7.71 (s, 2 H); LRMS APCl m/z 439 [M + H]⁺ | 57% |
| 26 | 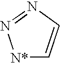 | CH₃ | ¹HNMR (400 MHz, DMSO-d₆) δ: 2.40 (s, 3 H),3.80 (s, 3 H), 5.60 (s, 2 H), 7.00 (m, 4 H), 7.25 (m,4 H), 7.50 (m, 4 H), 7.70 (s, 1 H), 7.80 (s, 1 H);LRMS APCl m/z 423 [M + H]⁺ | 44% |
| 27 | 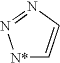 | OCH₃ | LRMS APCl m/z 439 [M + H]⁺ | 73% |

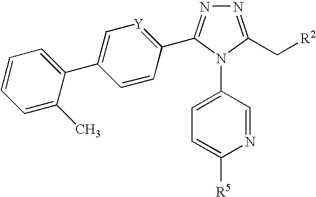

Y = CH

| | | | | |
|---|---|---|---|---|
| 28 | 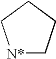 | OCH₃ | ¹HNMR (400 MHz, CDCl₃) δ: 1.80 (m, 4 H),2.20 (s, 3 H), 2.60 (m, 4 H), 3.60 (s, 2 H), 4.00 (s,3 H), 6.80 (d, 1 H), 7.20 (m, 6 H), 7.50 (d, 2 H),7.60 (d, 1 H), 8.20 (s, 1 H); LRMS APCl m/z 426[M + H]⁺ | 71% |

Y = N

| | | | | |
|---|---|---|---|---|
| 29 | 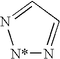 | OCH₃ | ¹HNMR (400 MHz, DMSO-d₆) δ: 2.20 (s, 3 H),4.00 (s, 3 H), 5.80 (s, 2 H), 6.70 (d, 1 H), 7.20 (d,1 H), 7.30 (m, 5 H), 7.40 (d, 1 H), 7.60 (s, 2 H),7.80 (d, 1 H), 8.30 (d, 1 H); LRMS APCl m/z 425[M + H]⁺ | 65% |
| 30 | 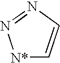 | OCH₃ | ¹HNMR (400 MHz, DMSO-d₆) δ: 2.20 (s, 3 H),4.00 (s, 3 H), 5.70 (s, 2 H), 6.80 (d, 1 H), 7.20 (d,1 H), 7.30 (m, 4 H), 7.80 (m, 3 H), 8.00 (s, 1 H),8.20 (m, 2 H); LRMS APCl m/z 425 [M + H]⁺ | 55% |
| 31 | 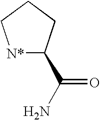 | OCH₃ | ¹HNMR (400 MHz, CDCl₃) δ: 1.80 (m, 4 H),2.20 (s, 3 H), 2.60 (m, 1 H), 3.10 (m, 1 H), 3.20 (m,1 H), 3.90 (d, 1 H), 4.00 (s, 3 H), 5.00 (s, 1 H),6.80 (s, 1 H), 7.20 (d, 1 H), 7.30 (m, 3 H), 7.60 (d,1 H), 7.80 (d, 1 H), 8.10 (s, 1 H), 8.30 (d, 2 H);LRMS APCl m/z 470 [M + H]⁺ | 65% |

### EXAMPLE 32

### 6-Methoxy-3-[3-(2′-methoxybiphenyl-4-yl)-5-methyl-4H-1,2,4-triazol-4-yl]-2-(2-methoxyethoxy)pyridine

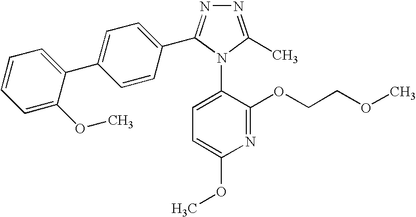

Sodium hydride (60% dispersion in mineral oil, 10 mg, 0.25 mmol) was added to a solution of 2-methoxyethanol (16 μL, 0.2 mmol) in tetrahydrofuran (5 mL) and the mixture was stirred for 10 minutes at room temperature. A solution of the product of preparation 56 (70 mg, 0.16 mmol) in tetrahydrofuran (2 mL) was added and the mixture was stirred for a further hour at room temperature. The reaction mixture was then concentrated in vacuo and the residue was partitioned between dichloromethane and sodium hydrogen carbonate solution. The aqueous solution was separated and extracted with dichloromethane (2×10 mL). The combined organic solution was then filtered through a phase separation cartridge and the filtrate was concentrated in vacuo and purified by column chromatography on silica gel, eluting with dichloromethane:methanol, 98:2, to afford the title compound as a white foam in 84% yield, 60 mg.

¹HNMR(400 MHz, CDCl₃) δ: 2.39(s, 3H), 3.26(s, 3H), 3.51-3.61(m, 2H), 3.79(s, 3H), 3.98(s, 3H), 4.43(m, 1H), 4.56(m, 1H), 6.40(d, 1H), 6.96(d, 1H), 7.00(m, 1H), 7.25-7.35(m, 3), 7.50(m, 4H); LRMS APCI m/z 447 [M+H]⁺

### EXAMPLE 33

### N-[2-({6-Methoxy-3-[3-(2′-methoxybiphenyl-4-yl)-5-methyl-4H-1,2,4-triazol-4-yl]pyridin-2-yl}oxy)ethyl]acetamide

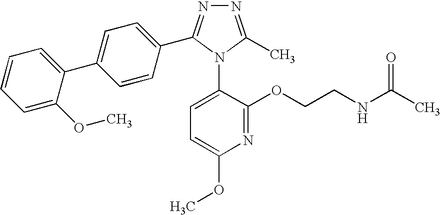

Sodium hydride (60% dispersion in mineral oil, 10 mg, 0.26 mmol) was added to a solution of N-(2-hydroxyethyl)acetamide (12 μL, 0.13 mmol) in tetrahydrofuran (2 mL) and the mixture was stirred for 15 minutes at room temperature. A solution of the product of preparation 56 (60 mg, 0.13 mmol) in tetrahydrofuran (2 mL) was added and the mixture was stirred for 2 hours at room temperature. Further sodium hydride (60% dispersion in mineral oil, 10 mg, 0.26 mmol) and N-(2-hydroxyethyl)acetamide (12 μL, 0.13 mmol) in tetrahydrofuran (2 mL) were then added and stirring continued for 18 hours at room temperature. The reaction mixture was quenched with sodium hydrogen carbonate solution and then concentrated in vacuo. The aqueous residue was extracted with dichloromethane (2×15 mL) and the combined organic solution was dried over sodium sulfate and concentrated in vacuo. Purification of the residue by column chromatography on silica gel, eluting with dichloromethane:methanol, 96:4, afforded the title compound as a white solid in 80% yield, 22 mg.

¹HNMR(400 MHz, CDCl₃) δ: 1.77(s, 3H), 2.33(s, 3H), 3.30-3.40(m, 2H), 3.77(s, 3H), 3.96(s, 3H), 4.14-4.22(m, 1H), 4.52-4.60(m, 1H), 5.43-5.48(m, 1H), 6.48(d, 1H), 6.95(d, 1H), 6.99(m, 1H), 7.24(d, 1H), 7.31 (m, 1H), 7.45-7.53(m, 5H); LRMS APCI m/z 474 [M+H]⁺

### EXAMPLE 34

### 2-Ethoxy-6-methoxy-3-[3-(2′-methoxybiphenyl-4-yl)-5-methyl-4H-1,2,4-triazol-4-yl]pyridine

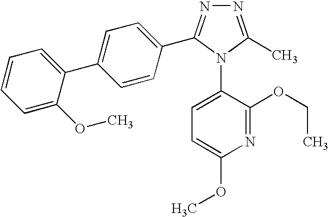

Sodium ethoxide (15 mg, 0.30 mmol) was added to a solution of the product of preparation 56 (60 mg, 0.13 mmol) in tetrahydrofuran (4 mL) and the mixture was stirred for 24 hours at room temperature. The reaction mixture was then concentrated in vacuo and the residue was purified by column chromatography on silica gel, eluting with dichloromethane:methanol, 98:2, to afford the title compound as a white solid in 33% yield, 25 mg.

¹HNMR(400 MHz, CDCl₃) δ: 1.22(t, 3H), 2.44(s, 3H), 3.79(s, 3H), 3.98(s, 3H), 4.38(q, 2H), 6.41(d, 1H), 6.97(d, 1H), 7.01 (t, 1H), 7.26(dd, 1H), 7.32(m, 1H), 7.40(d, 1H), 7.48-7.52(m, 4H); LRMS APCI m/z 417[M+H]⁺

### EXAMPLE 35

### 2-8 2-(3-Biphenyl-4-yl-5-methyl-4H-1,2,4-triazol-4-yl)-5-chlorophenoxy]-N,N-dimethylacetamide

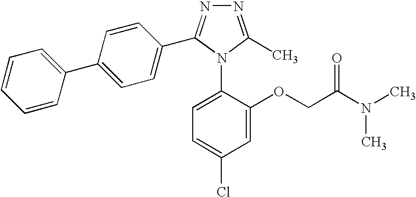

A mixture of the products of preparations 40 (180 mg, 0.5 mmol), 39 (152 mg, 0.75 mmol) and potassium carbonate (210 mg, 1.5 mmol) in acetonitrile (3 mL) and N,N-dimethylformamide (3 mL) was stirred at room temperature for 72 hours. The reaction mixture was then diluted with water and ethyl acetate, and the aqueous layer was separated and extracted with ethyl acetate (×3). The combined organic solution was washed with brine, dried over sodium sulfate and concentrated in vacuo to give an orange oil. This oil was purified by column chromatography on silica gel, eluting with dichloromethane:methanol:0.88 ammonia, 98:2:0.2. The resulting residue was triturated with diethyl ether to afford the title compound as a white solid in 25% yield, 56 mg.

¹HNMR(400 MHz, CDCl₃) δ: 2.37(s, 3H), 2.86(s, 3H), 2.92(s, 3H), 4.69(s, 2H), 7.03-7.10(m, 3H), 7.32-7.58(m, 9H); LRMS APCI m/z 447 [M+H]⁺

The following preparations illustrate the synthesis of certain intermediates used in the preparation of the preceding examples:

Preparation 1

### 5-Bromopyridine-2-carbohydrazide

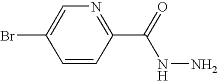

5-Bromo-pyridine-2-carboxylic acid methyl ester [(18.10 g, 83 mmol) J. Org. Chem., 2001, 66, 605] and hydrazine monohydrate (12.5 mL, 250 mmol) were dissolved in methanol (400 mL) and the mixture was heated under reflux for 48 hours. The reaction mixture was then filtered and the residue was dried in vacuo to afford the title compound as a solid in 86% yield, 15.40 g.

¹HNMR(DMSO-D₆, 400 MHz) δ: 4.57(d, 2H), 7.91(m, 1H), 8.22(m, 1H), 8.72(m, 1H), 9.98(m, 1H); LRMS ES+ m/z 217 [M+H]⁺

Preparation 2

### 4-Bromo-N′-(chloroacetyl)benzohydrazide

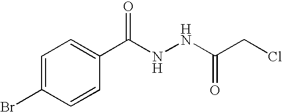

4-Methylmorpholine N-oxide (17.6 g, 174 mmol) and chloroacetyl chloride (15.7 g, 139 mmol) were added to a suspension of 4-bromobenzohydrazide (25 g, 116 mmol) in dichloromethane (350 mL) and the mixture was stirred at room temperature for 90 minutes. The reaction mixture was filtered and the residue was triturated with dichloromethane. Further trituration with water then afforded the title compound as a solid in 76% yield, 25.7 g.

¹HNMR(400 MHz, DMSO-d₆) δ: 4.10(s, 2H), 7.70(d, 2H), 7.80(d, 2H), 10.40(s, 1H), 10.60(d, 1H)

Preparation 3

### 5-Bromo-N′-(chloroacetyl)pyridine-2-carbohydrazide

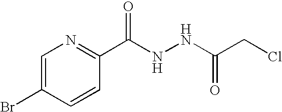

The title compound was prepared from the product of preparation 1 and chloroacetyl chloride, using the same method as that described for preparation 21, as a white solid in 67% yield.

¹HNMR(400 MHz, DMSO-d₆) δ: 4.15(s, 2H), 7.92(d, 1H), 8.25(d, 1H), 8.79(s, 1H), 10.40(s, 1H), 10.69(s, 1H); LRMS APCI m/z 293 [M+H]⁺

Preparation 4

### 5-Bromo-2-(5-methyl-1,3,4-oxadiazol-2-yl)pyridine

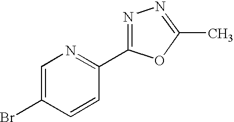

A solution of the product of preparation 1 (10 g, 46.3 mmol) and N,N-dimethylacetamide dimethyl acetal (10 mL, 68.4 mmol) in N,N-dimethylformamide (75 mL) was heated at 60° C. for 2.5 hours. The solution was concentrated in vacuo and the residue taken up in toluene (100 mL) and treated with para-toluenesulfonic acid monohydrate (500 mg). The mixture was heated to reflux for 3 hours, allowed to cool, and the resulting precipitate was filtered off, yielding an orange solid. The filtrate was concentrated in vacuo and the residue was combined with the orange solid and purified by column chromatography on silica gel, eluting with dichloromethane to afford the title compound as a white solid in 47% yield, 5.20 g.

¹HNMR(400 MHz, CDCl₃) δ: 2.65(s, 3H), 8.01(m, 1H), 8.12(m, 1H), 8.80(m, 1H); LRMS APCI m/z 240 [M+H]⁺

Preparation 5

### 6-Chloro-N-(methoxyacetyl)nicotinohydrazide

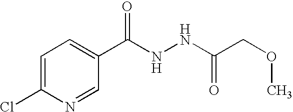

The title compound was prepared from 6-chloronicotinohydrazide and methoxyacetyl chloride, using the same method as that described for preparation 2, as a beige solid in 90% yield.

¹HNMR(CDCl₃, 400 MHz) δ: 3.36(s, 3H), 3.97(s, 2H), 7.68(d, 1H), 8.26(dd, 1H), 9.99(s, 1H), 10.61 (s, 1H). LRMS ESI m/z 244/246 [M+H]⁺

Preparation 6

### 2-(4-Bromophenyl)-5-(chloromethyl)-1,3,4-oxadiazole

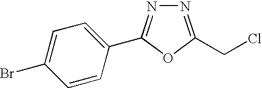

The product of preparation 2 (25.5 g, 87 mmol) was added to phosphorous oxychloride (90 mL) and the mixture was heated at 110° C. for 3 hours. The reaction mixture was then concentrated in vacuo and the residue was diluted with water. The aqueous mixture was basified using sodium hydrogen carbonate solution and extracted with ethyl acetate (500 mL). The organic solution was washed with water (500 mL) and brine (250 mL), dried over sodium sulfate and concentrated in vacuo to afford the title compound in 77% yield, 18.41 g.

¹HNMR(400 MHz, CDCl₃) δ: 4.80(s, 2H), 7.70 (d, 2H), 8.80(d, 2H); Microanalysis: C₉H₆BrClO requires (%): C, 39.52; H, 2.21; N, 10.24; found (%): C, 39.35; H, 2.27, N, 10.17.

Preparation 7

### 5-Bromo-2-[5-(chloromethyl)-1,3,4-oxadiazol-2-yl]pyridine

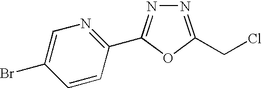

The title compound was prepared from the product of preparation 3, using the same method as that described for preparation 6. The crude compound was triturated with diethyl ether to afford the desired product as an off white solid in 57% yield.

¹HNMR(DMSO-D₆, 400 MHz) δ: 4.80(s, 2H), 8.05(d, 1H), 8.15(d, 1H), 8.85(s, 1H); LRMS APCI m/z 276 [M+H]⁺

Preparation 8

### 2-Chloro-5-[5-(methoxymethyl)-1,3,4-oxadiazol-2-yl]pyridine

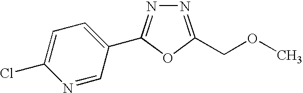

The product of preparation 5 (19 g, 78 mmol) was added to phosphorous oxychloride (70 mL) and the mixture was heated at 120° C. for 90 minutes. The reaction mixture was cooled to room temperature, concentrated in vacuo and the residue was partitioned between sodium carbonate solution and ethyl acetate. The aqueous solution was separated and extracted with dichloromethane (×4), and the combined organic solution was dried over magnesium sulfate and concentrated in vacuo. Re-crystallisation of the residue from ethyl acetate then afforded the title compound as a brown crystalline solid in 40% yield, 7.93 g.

¹HNMR(CDCl₃, 400 MHz) δ: 3.52(s, 3H), 4.74(s, 2H), 7.50(d, 1H), 8.32(dd, 1H), 9.06(d, 1H); LRMS APCI+m/z 226 [M+H]⁺Microanalysis: C₉H₈ClN₃O₂ requires (%): C, 47.91; H, 3.57; N, 18.62; found (%): C, 47.75; H, 3.50, N 18.46.

Preparation 9

### 2-(4-Bromophenyl)-5-(methoxymethyl)-1,3,4-oxadiazole

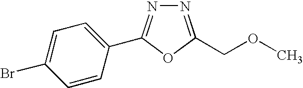

A solution of sodium methoxide (491 mg, 3.8 mmol) in methanol (5 mL) was added to a solution of the product of preparation 6 (700 mg, 2.6 mmol) in methanol (5 mL) and the mixture was stirred at room temperature for 20 hours. The reaction mixture was then concentrated in vacuo, and the residue was dissolved in water with a few drops of methanol. The aqueous solution was then extracted with dichloromethane and ethyl acetate, and the combined organic solution was dried over sodium sulfate and concentrated in vacuo to afford the title compound in 71% yield, 500 mg.

¹HNMR(400 MHz, CDCl₃) δ: 3.49(s, 3H), 4.71(s, 2H), 7.65(d, 2H), 7.93(d, 2H); LRMS APCI m/z 269/271 [M+H]⁺

Preparation 10

### 2-(1H-imidazol-1-ylmethyl)-5-(4-iodophenyl)-1,3,4-oxadiazole

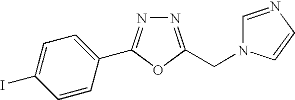

Potassium carbonate (860 mg, 6.24 mmol) was added to a mixture of 2-(chloromethyl)-5-(4-iodophenyl)-1,3,4-oxadiazole [(1 g, 3.12 mmol) WO 03/93248, p 22] and imidazole (234 mg, 3.43 mmol) in dimethylsulfoxide (5 mL) and the mixture was stirred for 20 hours at room temperature. The reaction mixture was then washed with water (3×50 mL) and the resulting precipitate was filtered off to afford the title compound as a brown solid in 58% yield, 639 mg.

¹HNMR(400 MHz, DMSO-d₆) δ: 5.60(s, 2H), 7.00(s, 1H), 7.30(s, 1H), 7.70(d, 2H), 7.80(s, 1H), 8.00(d, 2H); LRMS APCI m/z 353 [M+H]⁺

Preparation 11

### 2-[(3,5-Dimethyl-1H-pyrazol-1-yl)methyl]-5-(4-iodophenyl)-1,3,4-oxadiazole

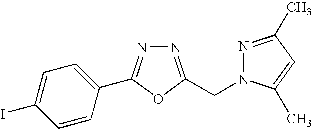

Potassium carbonate (862 mg, 6.24 mmol) was added to a mixture of 2-(chloromethyl)-5-(4-iodophenyl)-1,3,4-oxadiazole [(1 g, 3.12 mmol) WO 03/93248, p 22] and 3,5-dimethylpyrazole (360 mg, 3.74 mmol) in dimethylsulfoxide (5 mL) and the mixture was stirred for 18 hours at room temperature and at 50° C. for 2 hours. The reaction mixture was then diluted with ethyl acetate and washed with water (2×50 mL). The aqueous solution was extracted with dichloromethane and the combined organic layer was dried over sodium sulfate and concentrated in vacuo. Re-crystallisation of the residue from methanol/ethyl acetate afforded the title compound in 92% yield, 1.10 g. LRMS APCI m/z 381 [M+H]⁺

Preparation 12

### 1-{[5-(5-Bromopyridin-2-yl)-1,3,4-oxadiazol-2-yl]methyl}-L-prolinamide

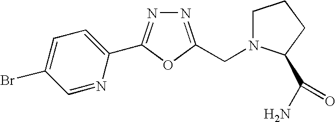

Potassium carbonate (503 mg, 3.64 mmol) was added to a mixture of the product of preparation 7 (500 mg, 1.82 mmol) and L-prolinamide (312 mg, 2.73 mmol) in acetonitrile (10 mL) and the mixture was stirred for 18 hours at room temperature and at 50° C. for 2 hours. The reaction mixture was concentrated in vacuo and the residue was diluted with ethyl acetate and washed with water (2×50 mL). The resulting precipitate was filtered off and the filtrate was washed with water, 2M sodium hydroxide solution and brine. The organic solution was then dried over sodium sulfate and concentrated in vacuo to afford the title compound.

¹HNMR(400 MHz, DMSO-d₆) δ: 1.70(m, 3H), 2.00(m, 1H), 2.60(m, 1H), 3.10(m, 1H), 3.20(m, 1H), 4.00(d, 1H), 4.20(d, 1H), 7.00-7.20(d, 2H), 8.10(d, 1H), 8.30(d, 1H), 8.90(s, 1H), LRMS APCI m/z 354 [M+H]⁺

Preparation 13

### 2-(4-Bromophenyl)-5-(1H-pyrazol-1-ylmethyl)-1,3,4-oxadiazole

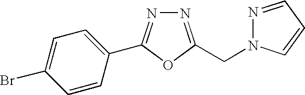

Potassium carbonate (860 mg, 6.24 mmol) was added to a mixture of the product of preparation 6 (1 g, 3.63 mmol) and pyrazole (299 mg, 4.40 mmol) in dimethylsulfoxide (5 mL) and the mixture was heated at 55° C. for 18 hours. The reaction mixture was then diluted with ethyl acetate and washed with water and brine. The organic solution was dried over sodium sulfate, concentrated in vacuo and the residue was re-dissolved in methanol, treated with charcoal and triturated with diethyl ether to afford the title compound as a pale brown solid in 73% yield, 804 mg.

¹HNMR(400 MHz, DMSO-d₆) δ: 5.60(s, 2H), 6.20(s, 1H), 7.60(m, 4H), 7.90(m, 2H); LRMS APCI m/z 305 [M+H]⁺

Preparation 14

### 2-(4-Bromophenyl)-5-[(2-methyl-1H-imidazol-1-yl)methyl]-1,3,4-oxadiazole

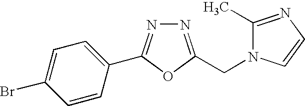

The title compound was prepared from the product of preparation 6 and 2-methyl-1H-imidazole, using the same method as that described for preparation 13, as a beige solid in 30% yield.

¹HNMR(400 MHz, CDCl₃) δ: 2.53(s, 3H), 5.33(s, 2H), 6.98-7.01 (m, 2H), 7.66(d, 2H), 7.85(d, 2H); LRMS APCI m/z 321 [M+H]⁺

Preparation 15

### 2-(4-Bromophenyl)-5-(pyrrolidin-1-ylmethyl)-1,3,4-oxadiazole

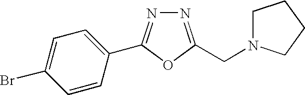

The title compound was prepared from the product of preparation 6 and pyrrolidine, using a similar method to that described for preparation 13. The crude compound was purified by column chromatography on silica gel, eluting with dichloromethane:methanol, 99:1 to 98:2 to afford the title compound in quantitative yield.

¹HNMR(400 MHz, CDCl₃) δ: 1.90(m, 4H), 2.70(m, 4H), 4.00(s, 2H), 7.65(d, 2H), 8.00(d, 2H); LRMS APCI m/z 310 [M+H]⁺

Preparations 16 and 17

Potassium carbonate (2.02 g, 14.62 mmol) was added to a mixture of the product of preparation 6 (2 g, 7.31 mmol) and 1,2,3-triazole (0.61 g, 8.77 mmol) in dimethylsulfoxide (5 mL) and the mixture was heated at 50° C. for 2 hours. The reaction mixture was then diluted with ethyl acetate and washed with water (×5). The aqueous solution was re-extracted with dichloromethane and the combined organic solution was dried over sodium sulfate and concentrated in vacuo. Purification of the residue by column chromatography on silica gel, eluting with dichloromethane:methanol, 100:0 to 98.8:1.2, afforded the product of preparation 16. Further elution, using the same gradient, then afforded the product of preparation 17.

Preparation 16

### 2-(4-Bromophenyl)-5-(2H-1,2,3-triazol-2-ylmethyl)-1,3,4-oxadiazole

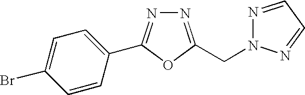

¹HNMR(400 MHz, CDCl₃) δ: 5.93(s, 2H), 7.62(d, 2H), 7.70(d, 2H), 7.89(m, 2H)

Yield=10%

Preparation 17

### 2-(4-Bromophenyl)-5-(1H-1,2,3-triazol-1-ylmethyl)-1,3,4-oxadiazole

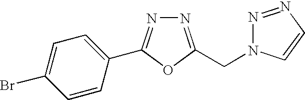

¹HNMR(400 MHz, CDCl₃) δ: 5.90(s, 2H), 7.65(d, 2H), 7.79(m, 2H), 7.85(d, 2H)

Yield=6%

Preparations 18 and 19

Potassium carbonate (755 mg, 5.46 mmol) was added to a mixture of the product of preparation 7 (750 mg, 2.73 mmol) and 1,2,3-triazole (0.61 g, 8.77 mmol) in N,N-dimethylformamide (5 mL) and the mixture was heated at 50° C. for 1 hour. The reaction mixture was then concentrated in vacuo and the residue was diluted with ethyl acetate (50 mL) and washed with water (50 mL). The organic solution was filtered off, yielding some crude title compound. The filtrate was washed with 10% citric acid and brine, dried over sodium sulfate and concentrated in vacuo. The residue was combined with the initial crop of crude product and the mixture was dissolved in methanol and treated with charcoal. Purification of the residue by column chromatography on silica gel, eluting with dichloromethane:methanol, 100:0 to 97:3, afforded the product of preparation 18. Further elution, using the same gradient, then afforded the product of preparation 19.

Preparation 18

### 5-Bromo-2-[5-(2H-1,2,3-triazol-2-ylmethyl)-1,3,4-oxadiazol-2-yl]pyridine

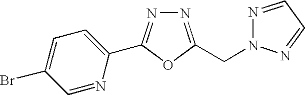

¹HNMR(400 MHz, CDCl₃) δ: 6.00(s, 2H), 7.70(s, 2H), 8.00(d, 1H), 8.10(d, 1H), 8.80(s, 1H); LRMS APCI m/z 308 [M+H]⁺

Yield=20%

Preparation 19

### 5-Bromo-2-[5-(1H-1,2,3-triazol-1-ylmethyl)-1,3,4-oxadiazol-2-yl]pyridine

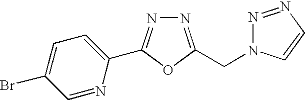

¹HNMR(400 MHz, CDCl₃) δ: 6.00(s, 2H), 7.80(s, 2H), 8.00(d, 1H), 8.10(d, 1H), 9.80(s, 1H); LRMS APCI m/z 308 [M+H]⁺

Yield=17%

Preparation 20

### 2-(4-Bromo-phenyl)-5-methyl-[1,3,4]oxadiazole

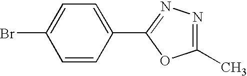

A solution of 4-bromo-benzoic acid hydrazide (12.90 g, 60 mmol) and N,N-dimethylacetamide dimethyl acetal (12 mL, 82.0 mmol) in N,N-dimethylformamide (100 mL) was heated at 60° C. for 2 hours. The solution was concentrated in vacuo and the residue taken up in toluene (80 mL) and treated with para-toluenesulfonic acid monohydrate (200 mg, 1.50 mmol). The mixture was heated to reflux for 2 hours, allowed to cool, and the resulting precipitate was filtered off. The crude product was washed with ether and dried in vacuo to yield a white solid. The filtrate was concentrated in vacuo and the residue was combined with the white solid, dissolved in toluene (50 mL) and treated with para-toluenesulfonic acid monohydrate (100 mg, 0.75 mmol). The mixture was heated under reflux for 3 hours, allowed to cool and concentrated in vacuo. Purification of residue by column chromatography on silica gel eluting with pentane:ethyl acetate, 80:20 to 40:60, afforded the title compound in 84% yield, 12.00 g.

¹HNMR(CDCl₃, 400 MHz) δ: 2.61(s, 3H), 7.62(d, 2H), 7.88(d, 2H); LRMS ESI m/z 239 [M+H]⁺

Preparation 21

### 5-[3-(4-Bromophenyl)-5-(methoxymethyl)-4H-1,2,4-triazol-4-yl]-2-methoxypyridine

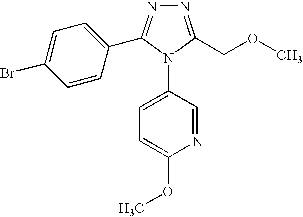

A mixture of the product of preparation 9 (480 mg, 1.78 mmol), 5-methoxypyridin-2-amine (250 mg, 1.96 mmol) and para-toluenesulfonic acid (50 mg, cat) in xylene (7 mL) was heated at 140° C. for 18 hours. The reaction mixture was then cooled to room temperature and directly purified by column chromatography on silica gel, eluting with ethyl acetate followed by dichloromethane:methanol:0.88 ammonia, 98:2:0.2. The appropriate fractions were evaporated under reduced pressure and the residue was triturated with diethyl ether to afford the title compound as a peach solid in 46% yield, 308 mg.

¹HNMR(400 MHz, CDCl₃) δ: 3.36(s, 3H), 4.48(s, 2H), 6.84(d, 1H), 7.34(d, 2H), 7.47(d, 3H), 8.10(s, 1H); LRMS ESI m/z 397/399 [M+Na]⁺; Microanalysis: C₁₆H₁₅BrN₄O requires: C, 51.22; H, 4.03; N, 14.93; found C, 51.14; H, 4.01, N, 14.89.

Preparation 22

### 3-(4-Bromophenyl)-4-(4-methoxyphenyl)-5-methyl-4H-1,2,4-triazole

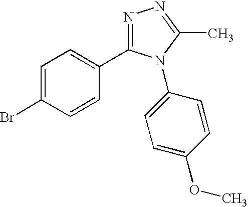

The title compound was prepared from the product of preparation 20 and 4-methoxyaniline, using the same method as that described for preparation 21. The crude compound was purified by column chromatography on silica gel eluting with dichloromethane:methanol:0.88 ammonia 100:0:0 to 97:3:0.3 to afford the desired product in 98% yield.

¹HNMR(DMSO-d₆, 400 MHz) δ: 2.20(s, 3H), 3.81(s, 3H), 7.07(m, 2H), 7.28(m, 2H), 7.34(m, 2H), 7.56(m, 2H). LRMS APCI m/z 344 [M+H]⁺

Preparation 23

### 3-(1H-Imidazol-1-ylmethyl)-5-(4-iodophenyl)-4-(4-methoxyphenyl)-4H-1,2,4-triazole

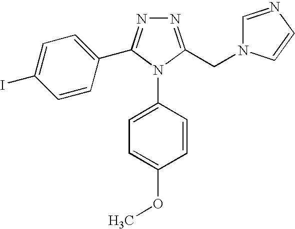

A mixture of the product of preparation 10 (310 mg, 1.03 mmol), 4-methoxyaniline (330 mg, 2.68 mmol) and para-toluenesulfonic acid (10 mg) in xylene (10 mL) was heated at 140° C. for 3 hours. Further para-toluenesulfonic acid (10 mg) was then added and heating continued at 140° C. for 2.5 hours. The cooled reaction mixture was concentrated in vacuo and the residue was dissolved in ethyl acetate (50 mL), washed with 2M sodium hydroxide solution (50 mL), water (50 mL) and brine (50 mL). The organic solution was dried over sodium sulfate and concentrated in vacuo and the residue was purified by column chromatography on silica gel, eluting with ethyl acetate followed by dichloromethane:methanol:0.88 ammonia, 97.5:2.5:0.25. The appropriate fractions were evaporated under reduced pressure and the residue was dissolved in methanol, treated with charcoal and concentrated in vacuo to afford the title compound in 44% yield, 179 mg.

¹HNMR(400 MHz, CDCl₃) δ: 3.90(s, 3H), 5.20(s, 2H), 6.90(m, 3H), 7.00(d, 1H), 7.10(d, 2H), 7.20(d, 1H), 7.60(d, 2H); LRMS APCI m/z 458 [M+H]⁺

Preparation 24

### 3-(1H-Imidazol-1-ylmethyl)-5-(4-iodophenyl)-4-(4-methylphenyl)-4H-1,2,4-triazole

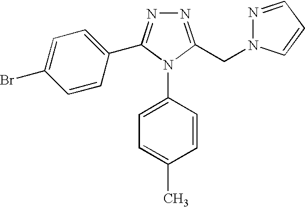

The title compound was prepared from the product of preparation 13 and 4-methylaniline, using the same method as that described for preparation 23, as a yellow oil in 95% yield.

¹HNMR(400 MHz, CDCl₃) δ: ¹HNMR(400 MHz, CDCl₃) δ: 2.40(s, 3H), 5.40(s, 2H), 6.80(d, 1H), 7.21-7.30(m, 6H), 7.39-7.42(m, 4H); LRMS APCI m/z 366 [M+H]⁺

Preparation 25

### 3-(4-Bromophenyl)-5-[(2-methyl-1H-imidazol-1-yl)methyl]-4-(4-methylphenyl)-4H-1,2,4-triazole

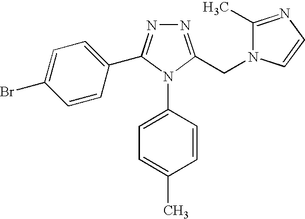

The title compound was prepared from the product of preparation 14 and 4-methylaniline, using the same method as that described for preparation 23, as a brown oil in 73% yield.

LRMS APCI m/z 409 [M+H]⁺

Preparation 26

### 3-(4-Bromophenyl)-4-(4-methoxyphenyl)-5-(1H-pyrazol-1-ylmethyl)-4H-1,2,4-triazole

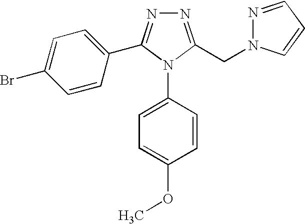

A mixture of the product of preparation 13 (240 mg, 0.79 mmol), 4-methoxyaniline (145 mg, 1.18 mmol) and para-toluenesulfonic acid (10 mg) in xylene (5 mL) was heated at 140° C. for 18 hours. The cooled reaction mixture was concentrated in vacuo and the residue was dissolved in ethyl acetate (50 mL), washed with sodium hydrogen carbonate solution (3×15 mL) and brine (20 mL). The organic solution was dried over magnesium sulfate, treated with charcoal and concentrated in vacuo to afford the title compound as a pale yellow oil in 81% yield, 261 mg.

¹HNMR(400 MHz, CDCl₃) δ: 3.85(s, 3H), 5.39(s, 2H), 6.24(s, 1H), 6.68(m, 1H), 6.74(m, 1H), 6.90(m, 3H), 7.30(m, 2H), 7.39-7.43(m, 3H); LRMS APCI m/z 412 [M+H]⁺

Preparations 27 to 36

The following compounds, of the general formula shown below, were prepared using the same method to that described for preparation 26, using the products of preparation 10, 11, 12 and 15-19, with commercially available amines

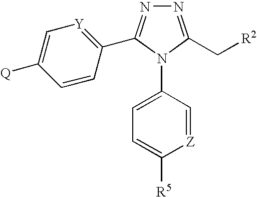

| | | | |
|---|---|---|---|
| No | R² | Data | Yield |

Y = CH, Z = CH

| | | | |
|---|---|---|---|
| 27 | 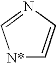 | Q = I, R⁵ = CH₃¹HNMR (400 MHz, CDCl₃) δ: 2.25 (s, 3 H), 3.80 (s, 3 H),5.20 (s, 2 H), 6.80 (m, 6 H), 7.00 (m, 3 H), 7.30 (m, 2 H),7.50 (m, 4 H); LRMS APCl m/z 422 [M + H]⁺ | 67% |
| 28 | 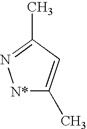 | Q = I, R⁵ = OCH₃LRMS APCl m/z 486 [M + H]⁺ | 92% |
| 29 | 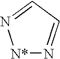 | Q = Br, R⁵ = CH₃LRMS APCl m/z 397 [M + H]⁺ | 72% |
| 30 | 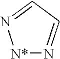 | Q = Br, R⁵ = OCH₃LRMS APCl m/z 411/413 [M + H]⁺ | 71% |
| 31 | 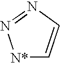 | Q = Br, R⁵ = CH₃LRMS APCl m/z 397 [M + H]⁺ | 78% |
| 32 | 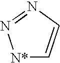 | Q = Br, R⁵ = OCH₃LRMS APCl m/z 413 [M + H]⁺ | 80% |

Z = N

| | | | |
|---|---|---|---|
| 33 | 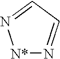 | Y = N, Q = Br, R⁵ = OCH₃¹HNMR (400 MHz, CDCl₃) δ: 4.00 (s, 3 H), 5.80 (s, 2 H),6.70 (d, 1 H), 7.30 (m, 1 H), 7.60 (s, 2 H), 7.90 (m, 2 H), 8.20 (d,1 H), 8.40 (s, 1 H); LRMS APCl m/z 415 [M + H]⁺ | 86% |
| 34 | 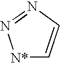 | Y = N, Q = Br, R⁵ = OCH₃¹HNMR (400 MHz, CDCl₃) δ: 4.00 (s, 3 H), 6.70 (s, 2 H),6.80 (d, 1 H), 7.30 (m, 1 H), 7.70 (d, 2 H), 7.90 (m, 2 H), 8.10 (d,1 H), 8.40 (s, 1 H); LRMS APCl m/z 415 [M + H]⁺ | 78% |
| 35 | 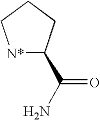 | Y = N, Q = Br, R⁵ = OCH₃¹HNMR (400 MHz, CDCl₃) δ: 1.80 (m, 2 H), 2.20 (m, 1 H),2.60 (m, 1 H), 3.10 (m, 1 H), 3.20 (m, 1 H), 3.90 (m, 2 H),4.00 (s, 3 H), 5.00 (s, 1 H), 6.70 (s, 1 H), 6.90 (d, 1 H), 7.60 (d,1 H), 7.90 (d, 1 H), 8.05 (d, 1 H), 8.20 (d, 1 H), 8.40 (s, 1 H);LRMS APCl m/z 458 [M + H]⁺ | 46% |
| 36 | 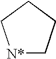 | Y = CH, Q = Br, R⁵ = OCH₃¹HNMR (400 MHz, CDCl₃) δ: 1.80 (m, 4 H), 2.60 (m, 4 H),3.60 (s, 2 H), 4.00 (s, 3 H), 6.80 (d, 1 H), 7.35 (d, 2 H), 7.45 (d,2 H), 7.50 (d, 1 H), 8.10 (s, 1 H); LRMS APCl m/z 414 [M + H]⁺ | 60% |

Preparation 35: Crude compound was purified by column chromatography on silica gel, eluting with dichloromethane:methanol:0.88 ammonia, dichloromethane:methanol, 100:0 to 94:6.

Preparation 36: Crude compound was purified by column chromatography on silica gel, eluting with dichloromethane:methanol:0.88 ammonia, 99:1:0.1 to 98:2:0.2.

Preparation 37

### (4-Chloro-2-methoxyphenyl)amine

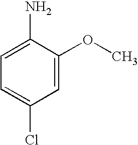

Sodium dithionite (28 g, 159 mmol) was added portionwise to a mixture of 5-chloro-2-nitroanisole (10 g, 53 mmol) and potassium hydrogen carbonate (28 g, 275.6 mmol) in methanol (150 mL) and water (150 mL) and the reaction mixture was stirred at room temperature for 2 hours. The mixture was then acidified with concentrated hydrochloric acid and the mixture was heated at 60° C. for 2 hours. The reaction mixture was then concentrated in vacuo and the aqueous residue was washed with ethyl acetate. The aqueous solution was then basified with sodium hydroxide and extracted with ethyl acetate. The organic solution was washed with brine, dried over sodium sulfate and concentrated in vacuo to afford the title compound in 64% yield, 5.35 g.

¹HNMR(400 MHz, DMSO-d₆) δ: 3.74(s, 3H), 4.78(s, 2H), 6.58(m, 1H), 6.65(m, 1H), 6.77(s, 1H)

Preparation 38

### 3-Biphenyl-4-yl-4-(4-chloro-2-methoxyphenyl)-5-methyl-4H-1,2,4-triazole

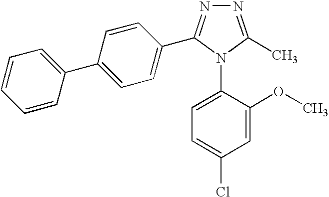

A mixture of 2-biphenyl-4-yl-5-methyl-1,3,4-oxadiazole [(1 g, 4.2 mmol), Bioorganic & Medicinal Chemistry, 2002, 10, 1905], the product of preparation 37 (800 mg, 5.04 mmol) and para-toluenesulfonic acid (200 mg) in xylene (20 mL) was heated at 150° C. for 10 hours. Further para-toluenesulfonic acid (100 mg) was added at regular intervals, until all of the starting material was consumed. After heating at 150° C. for 4.5 hours, the reaction mixture was diluted with 2M hydrochloric acid, extracted with ethyl acetate and concentrated in vacuo. The residue was purified by column chromatography on silica gel, eluting with dichloromethane:methanol:0.88 ammonia, 98:2:0.2. The appropriate fractions were evaporated under reduced pressure and the residue was triturated with ethyl acetate (×3) to afford the title compound as a pale brown solid in 51% yield, 860 mg.

¹HNMR(400 MHz, CDCl₃) δ: 2.30(s, 3H), 3.74(s, 3H), 7.04-7.10(m, 3H), 7.32-7.57(m, 9H); LRMS APCI m/z 376 [M+H]⁺

Preparation 39

### 2-(3-Biphenyl-4-yl-5-methyl-4H-1,2,4-triazol-4-yl)-5-chlorophenol

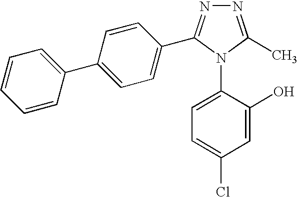

Boron tribromide (1M in dichloromethane, 1.6 mL, 1.6 mmol) was added to the product of preparation 38 (200 mg, 0.53 mmol) in dichloromethane (2 mL) and the mixture was heated under reflux for 4 hours. The mixture was then poured onto ice, basified with 2M sodium hydroxide and then acidified to pH5 with concentrated hydrochloric acid. The mixture was then extracted with dichloromethane (×3) and the combined organic solution was washed with brine, dried over sodium sulfate and concentrated in vacuo to afford the title compound as a beige solid in 96% yield, 183 mg.

¹HNMR(400 MHz, CDCl₃) δ: 2.10(s, 3H), 6.71-6.81 (m, 2H), 7.19-7.32(m, 12H); LRMS APCI m/z 260 [M+H]⁺

Preparation 40

### 2-Bromo-N,N-dimethylacetamide

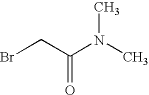

Triethylamine (4.4 mL, 33.3 mmol) was added to a mixture of bromoacetyl chloride (1.05 mL, 12.7 mmol) and dimethylamine hydrochloride (1.25 g, 15.2 mmol) in dichloromethane and the mixture was stirred for 2 hours at room temperature. The mixture washed with 2M hydrochloric acid, 2M sodium hydroxide solution and brine, dried over sodium sulfate and concentrated in vacuo to afford the title compound as a yellow oil in 38% yield, 800 mg.

¹HNMR(400 MHz, CDCl₃) δ: 2.98(s, 3H), 3.09(s, 3H), 4.07(s, 2H); LRMS APCI m/z 168 [M+H]⁺

Preparation 41

### 4-Bromo-2-methoxybenzoic Acid

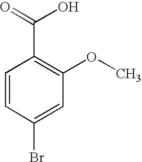

Methyl iodide (861 μL, 13.83 mmol) was added to a mixture of 4-bromo-2-hydroxybenzoic acid [(1.2 g, 5.53 mmol), J. Med. Chem. 1997, 40, 2843] and potassium carbonate (2.3 g, 16.59) in N,N-dimethylformamide (30 mL) and the mixture was stirred at room temperature for 18 hours. The reaction mixture was then concentrated in vacuo and the residue was partitioned between ethyl acetate and water. The aqueous was separated and extracted with ethyl acetate (×2) and the combined organic solution was washed with brine dried over sodium sulfate and concentrated in vacuo to afford the title compound as an orange oil in 94% yield, 1.27 g.

¹HNMR(400 MHz, CDCl₃) δ: 3.88(s, 1H), 3.90(s, 3H), 7.11-7.15(m, 2H), 7.68(d, 1H); LRMS APCI m/z 245/247 [M+H]⁺

Preparation 42

### 4-Bromo-2-methoxybenzohydrazide

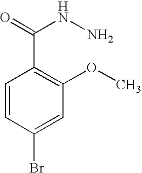

The title compound was prepared from the product of preparation 41 and hydrazine monohydrate, using a similar method to that of preparation 1. The crude compound was re-crystallised from methanol to afford the title compound as a solid in 79% yield.

¹HNMR(400 MHz, DMSO-d₆) δ: 3.85(s, 3H), 4.52(bs, 2H), 7.21(m, 1H), 7.30(m, 1H), 7.56(m, 1H); LRMS APCI m/z 245/247 [M+H]⁺

Preparation 43

### 2-(4-Bromo-2-methoxyphenyl)-5-methyl-1,3,4-oxadiazole

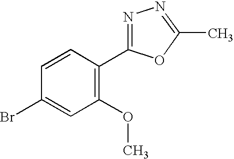

A solution of the product of preparation 42 (800 mg, 3.26 mmol) and N,N-dimethylacetamide dimethyl acetal (716 μL, 4.89 mmol) in N,N-dimethylformamide (15 mL) was heated at 60° C. for 2 hours. The reaction mixture was stirred at room temperature for 18 hours and then concentrated in vacuo. The residue taken up in toluene (20 mL), treated with para-toluenesulfonic acid monohydrate (100 mg) and heated under reflux for 5 hours. The mixture was then cooled to room temperature, concentrated in vacuo and the residue was partitioned between ethyl acetate and sodium hydrogen carbonate solution. The aqueous solution was separated, extracted with ethyl acetate (×2) and the combined organic solution was washed with brine, dried over sodium sulfate and concentrated in vacuo. Purification of the residue by column chromatography on silica gel, eluting with dichloromethane:methanol:0.88 ammonia, 100:0:0 to 99:1:0.1, afforded the title compound as an off-white solid in 85% yield, 760 mg.

¹HNMR(400 MHz, CDCl₃) δ: 2.62(s, 3H), 3.97(s, 3H), 7.20-7.23(m, 2H), 7.77(d, 1H); LRMS APCI m/z 269/271 [M+H]⁺

Preparation 44

### 3-(4-Bromo-2-methoxyphenyl)-4-(4-methoxyphenyl)-5-methyl-4H-1,2,4-triazole

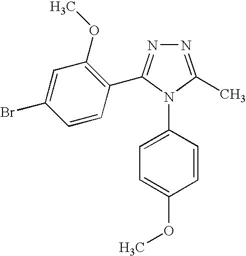

The title compound was prepared from the product of preparation 43 and 4-methoxyaniline, using a similar method as that described for preparation 25. The crude compound was twice purified by column chromatography on silica gel, eluting with dichloromethane:methanol:0.88 ammonia, 100:0:0 to 98:2:0.2, to afford the title compound as a dark pink foam in 82% yield, 798 mg.

¹HNMR(400 MHz, CDCl₃) δ: 2.50(s, 3H), 3.47(s, 3H), 3.82(s, 3H), 6.88-6.92(m, 3H). 7.00-7.04(m, 2H), 7.25-7.27(m, 2H); LRMS APCI m/z 374/376 [M+H]⁺

Preparation 45

### di-μ-Chlorobis[2-[(dimethylamino-κN)methyl]phenyl-κC]di-palladium

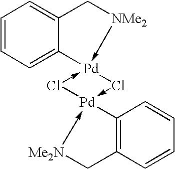

N,N-dimethylbenzylamine (5.82 mL, 38.7 mmol) was added to a suspension of palladium chloride (3.43 g, 19.4 mmol) in methanol (200 mL) and the resulting red/brown suspension was stirred at room temperature for 24 hours. The now green/brown suspension was concentrated in vacuo, and the residue was re-dissolved in dichloromethane (150 mL) and passed through a pad of silica gel; washing through with dichloromethane. The filtrate was concentrated in vacuo and the residue was re-crystallised from dichloromethane:ether to give the desired product, 4.66 g.

¹HNMR(CDCl₃, 300 MHz) δ: 2.86(s, 6H), 2.89(s, 6H), 3.95(s, 4H), 6.84-7.24(m, 8H)

Preparation 46

### bis[2-[(Dimethylamino-κN)methyl]phenyl-κC]bis[μ-(trifluoroacetato-κO: κO′)]-palladium

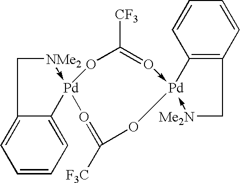

A solution of the complex of preparation 45 (5.60 g, 10.15 mmol) in dichloromethane (100 mL) was added to a solution of silver trifluoroacetate (4.48 g, 20.3 mmol) in acetone (30 mL). A thick white precipitate appeared during addition. The suspension was stirred for 15 minutes, and was then filtered through a pad of silica gel, washing through with dichloromethane. Concentration in vacuo gave a bright yellow powder that was re-crystallised from dichloromethane:ether to afford the title compound 7.06 g.

¹HNMR(CDCl₃, 300 MHz) δ: 2.05(s, 6H), 2.88(s, 6H), 3.18(d, 2H), 3.63(d, 2H), 6.89-6.97(m, 6H), 7.00-7.10(m, 2H)

Preparation 47

### [2-[(Dimethylamino-κn)methyl]phenyl-κc](tricyclohexylphosphine)(trifluoroacetato-κo-(sp-4-3)-palladium

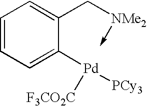

A solution of tricyclohexylphosphine (6.89 g, 24.5 mmol) in dichloromethane (20 mL) was added to a solution of the product of preparation 46 (6.43 g, 9.10 mmol) in dichloromethane (50 mL) and the mixture was stirred for 1 hour. The reaction mixture was then passed through a pad of silica gel, washing through with dichloromethane (400 mL), and the pale yellow filtrate was concentrated in vacuo. Re-crystallisation of the residue from dichloromethane:ether afforded the desired complex, 10.53 g.

¹HNMR(CDCl₃, 300 MHz) δ: 1.05-2.30(m, 33H), 2.57(s, 3H), 2.58(s, 3H), 3.93(s, 2H), 6.86-6.98(m, 3H), 7.10-7.12(m, 1H)

Preparation 48

### 2-(2′-Methoxybiphenyl-4-yl)-5-methyl-1,3,4-oxadiazole

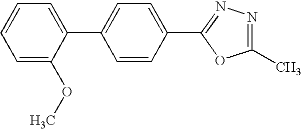

A mixture of the product of preparation 20 (1.1 g, 4.60 mmol), (2-methoxyphenyl)boronic acid (1.05 g, 6.90 mmol), caesium carbonate (4.48 g, 13.8 mmol) and the palladium complex of preparation 47 (3 mg) in dioxane (46 mL) was heated under reflux for 18 hours. The reaction mixture was then concentrated in vacuo and the residue was taken up in dichloromethane and washed with dilute hydrochloric acid. The organic solution was then washed with 1 M sodium hydroxide solution, dried over sodium sulfate and concentrated in vacuo. Purification of the residue by column chromatography on silica gel, eluting with pentane:diethyl ether, 80:20 to 30:70, afforded the title compound in 20% yield, 243 mg.

¹HNMR(CDCl₃, 400 MHz) δ: 2.63(s, 3H), 3.84(s, 3H), 7.00(d, 1H), 7.04(d, 1H), 7.34(d, 1H), 7.35(m, 1H), 7.66(d, 2H), 8.04(d, 2H); LRMS ESI m/z 267 [M+H]⁺

Preparation 49

### 5-[5-(Methoxymethyl)-1,3,4-oxadiazol-2-yl]-2-(2-methylphenyl)pyridine

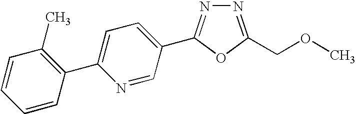

A mixture of the product of preparation 8 (500 mg, 2.22 mmol), (2-methylphenyl)boronic acid (361 mg, 2.66 mmol), caesium carbonate (2.16 g, 6.66 mmol) and the palladium complex of preparation 47 (10 mg) in dioxane (10 mL) was heated under reflux for 2 hours then stirred at room temperature for 18 hours. Further palladium complex (10 mg) (preparation 47) was added and mixture was stirred at room temperature for 1 hour. The reaction mixture was then concentrated in vacuo and the residue was partitioned between ethyl acetate and water. The organic layer was separated, washed with brine, dried over sodium sulfate and concentrated in vacuo to afford the title compound as a brown solid in quantitative yield.

¹HNMR(400 MHz, CDCl₃) δ: 2.34(s, 3H), 3.46(s, 3H), 4.69(s, 2H), 7.27-7.31(m, 3H), 7.39(m, 1H), 7.51(d, 1H), 8.37(dd, 1H), 9.30(m, 1H); LRMS APCI m/z 282 [M+H]⁺

Preparation 50

### 5-(2-Methoxyphenyl)-2-(5-methyl-1,3,4-oxadiazol-2-yl)pyridine

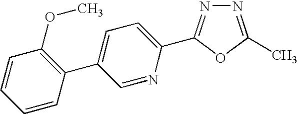

A mixture of the product of preparation 3 (200 mg, 0.85 mmol), (2-methoxyphenyl)boronic acid (290 mg, 1.91 mmol), caesium carbonate (1.25 g, 3.84 mmol) and the palladium complex of preparation 47 (10 mg) in dioxane (15 mL) was heated under reflux for 2.5 hours. Further palladium complex (10 mg) (preparation 47) was added and mixture was stirred at room temperature for 2 hours. Further palladium complex (10 mg) (preparation 47) was added and the mixture was heated under reflux for 2 hours. The cooled reaction mixture was then filtered through Celite®, washing through with dichloromethane and the filtrate was concentrated in vacuo. Purification of the residue by column chromatography on silica gel, eluting with dichloromethane:methanol:0.88 ammonia, 100:0:0 to 99:1:0.1, then afforded the title compound as a white solid in 50% yield, 110 mg.

¹HNMR(400 MHz, CDCl₃) δ: 2.67(s, 3H), 3.84(s, 3H), 7.03(m, 1H), 7.09(m, 1H), 7.35-7.44(m, 2H), 8.05(m, 1H), 8.26(m, 1H), 8.91 (m, 1H); LRMS APCI m/z 268 [M+H]⁺

Preparation 51

### 6-Methoxy-2-(methylthio)-3-nitropyridine

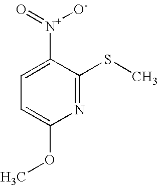

Sodium methanethiolate (2.59 g, 37 mmol) was added to an ice-cold suspension of 2-chloro-6-methoxy-3-nitropyridine (7.26 g, 38.5 mmol) in tetrahydrofuran (20 mL) and the mixture was stirred for 5 hours at room temperature. Further sodium methanethiolate (1.3 g, 18.5 mmol) was added and the mixture was stirred for an additional 18 hours at room temperature. The reaction mixture was then diluted with dichloromethane (250 mL), washed with water (250 mL), dried over sodium sulfate and concentrated in vacuo to afford the title compound in 94% yield, 7.23 g.

¹HNMR(400 MHz, CDCl₃) δ: 2.59(s, 1H), 4.08(s, 1H), 6.52(d, 1H), 8.41(d, 1H); LRMS APCI m/z 201 [M+H]⁺

Preparation 52

### 6-Methoxy-2-(methylthio)pyridin-3-amine

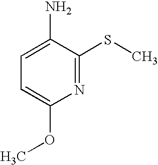

A mixture of Iron powder (4 g), ammonium chloride (2.0 g, 36.5 mmol) and the product of preparation 51 (7.2 g, 36 mmol) in ethanol (70 mL) was heated under reflux for 3 hours. The reaction mixture was then filtered through Celite® and the filtrate was concentrated in vacuo to afford the title compound as a purple solid in quantitative yield, 6.12 g.

¹HNMR(400 MHz, CDCl₃) δ: 2.62(s, 3H), 3.90(s, 3H), 6.63(d, 1H), 6.91(d, 1H); LRMS APCI m/z 171 [M+H]⁺

Preparation 53

### Ethyl-N-[6-methoxy-2-(methylthio)pyridin-3-yl]ethanimidoate

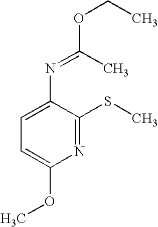

A mixture of the product of preparation 52 (5.95 g, 35 mmol) and triethylorthoacetate (12.8 mL, 70 mmol) was heated at 105° C. for 1 hour and 120° C. for 30 minutes. The reaction mixture was then concentrated in vacuo and the residue was taken up in pentane/diethyl ether, 90:10, and filtered through a pad of silica, washing through with further pentane/diethyl ether, 90:10. The filtrate was concentrated in vacuo to afford the title compound as a yellow oil in 76% yield, 6.4 g.

¹HNMR(400 MHz, CDCl₃) δ: 1.38(t, 3H), 1.82(s, 3H), 2.53(s, 3H), 3.95(s, 3H), 4.32(q, 2H), 6.38(d, 1H), 6.84(d, 1H); LRMS ESI m/z 241 [M+H]⁺

Preparation 54

### 3-[3-(4-Bromophenyl)-5-methyl-4H-1,2,4-triazol-4-yl]-6-methoxy-2-(methylthio)pyridine

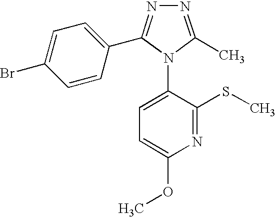

A mixture of the product of preparation 53 (6.4 g, 26.7 mmol) and 4-bromobenzohydrazide (4.58 g, 21.3 mmol) in N,N-dimethylformamide (15 mL) was heated at 120° C. for 18 hours. para-Toluenesulfonic acid (20 mg) was then added and heating continued at 120° C. for a further 3 hours. The reaction mixture was concentrated in vacuo and the residue was dissolved in dichloromethane (300 mL), washed with water (200 mL), dried over sodium sulfate and concentrated in vacuo. Trituration of the residue in hot dichloromethane afforded some title compound as a white solid, 2.93 g. The filtrate was the concentrated in vacuo and the residue was purified by column chromatography on silica gel, eluting with dichloromethane:methanol, 100:0 to 95:5, to afford further title compound (1.34 g), providing a total yield of 51%, 4.27 g.

¹HNMR(400 MHz, CDCl₃) δ: 2.30(s, 3H), 2.54(s, 3H), 4.05(s, 3H), 6.51(d, 1H), 7.29(d, 1H), 7.36(d, 2H), 7.43(d, 2H); LRMS ESI m/z 805 [2M+Na]⁺

Preparation 55

### 6-Methoxy-3-[3-(2′-methoxybiphenyl-4-yl)-5-methyl-4H-1,2,4-triazol-4yl]-2-(methylthio)pyridine

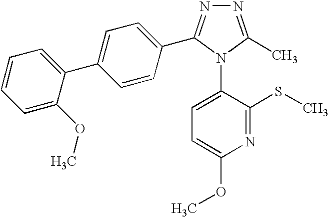

A mixture of the product of preparation 54 (2.5 g, 6.4 mmol), (2-methoxyphenyl)boronic acid (1.5 g, 9.6 mmol), sodium carbonate (1.3 g, 12.8 mmol) and tetrakis(triphenylphosphine)palladium(0) (60 mg) in 1,2-dimethoxyethane (50 mL) and water (6 mL) was heated under reflux for 2.5 hours. The reaction mixture was then concentrated in vacuo and the residue was diluted with dichloromethane (150 mL), washed with water (100 mL), dried over sodium sulfate and concentrated in vacuo. Purification of the residue by column chromatography on silica gel, eluting with dichloromethane:methanol, 100:0 to 95:5, afforded the title compound as a white foam in 92% yield, 2.46 g.

¹HNMR(400 MHz, CDCl₃) δ: 2.33(s, 3H), 2.55(s, 3H), 3.78(s, 3H), 4.05(s, 3H), 6.52(d, 1H), 6.96(d, 1H), 7.00(m, 1H), 7.23-7.36(m, 3H), 7.49(d, 2H), 7.54(d, 2H); LRMS APCI m/z 419 [M+H]⁺

Preparation 56

### 6-Methoxy-3-[3-(2′-methoxybiphenyl-4-yl)-5-methyl-4H-1,2,4-triazol-4yl]-2-(methylsulfonyl)pyridine

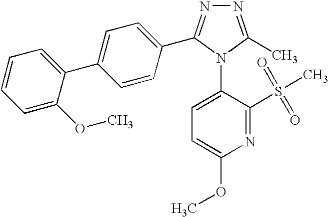

A mixture of the product of preparation 55 (4.2 g, 10.9 mmol) and meta-chloroperbenzoic acid (3.96 g, 22.9 mmol) in dichloromethane (60 mL) was stirred at room temperature for 18 hours. The reaction mixture was then washed with 10% sodium metabisulf ite (100 mL) and sodium hydrogen carbonate solution (2×100 mL) and the organic solution was dried over sodium sulfate and concentrated in vacuo. The residue was purified by column chromatography on silica gel, eluting with dichloromethane:methanol, 100:0 to 96:4, to afford the title compound as a white solid in 24% yield, 1.17 g.

¹HNMR(400 MHz, CDCl₃) δ: 2.42(s, 3H), 3.14(s, 3H), 3.77(s, 3H), 4.01 (s, 3H), 6.96(d, 1H), 6.99(d, 1H), 7.15(d, 1H), 7.25(dd, 1H), 7.31 (m, 1H), 7.49(d, 2H), 7.51 (d, 2H), 7.81 (d, 1H); LRMS APCI m/z 451 [M+H]⁺

Preparation 57

### Methyl-(5-nitro-pyridin-2-yl)-amine

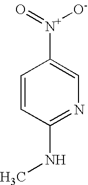

Methylamine gas was bubbled through a stirred solution of 2-chloro-5-nitropyridine (4 g, 25.2 mmol) in dichloromethane (60 mL), at room temperature, until saturation had occurred. The resulting yellow precipitate was then filtered off, washed with dichloromethane and dried under vacuum to afford the title compound as a yellow solid in 80% yield, 3.07 g.

¹HNMR(CDCl₃, 400 MHz) δ: 3.05(d, 3H), 5.41(bs, 1H), 6.37(d, 1H), 8.21(d, 1H), 9.03(d, 1H); LRMS APCI m/z 154 [M+H]⁺

Preparation 58

### N²-Methylpyridine-2,5-diamine

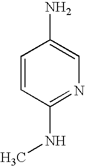

The product of preparation 57 (3.0 g, 19.5 mmol) and 10% Pd/C (300 mg) were stirred in ethanol (150 mL) under 60 psi of hydrogen gas for 18 hours. The reaction mixture was then filtered through Celite® and the filtrate was concentrated in vacuo to afford the title product in 17% yield, 400 mg.

¹HNMR(CDCl₃, 400 MHz) δ: 2.87(s, 3H), 3.19(bs, 2H), 4.18(bs, 1H), 6.31(d, 1H), 6.97(d, 1H); 7.68(d, 1H); LRMS ESI m/z 247 [2M+H]⁺

Preparation 59

### 4-{2-[(6-Methoxy-3-nitropyridin-2-yl)oxy]ethyl}morpholine

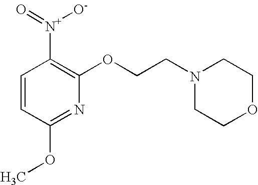

A solution of 4-(2-hydroxyethyl)morpholine (1.57 g, 12 mmol) in tetrahydrofuran (20 mL) was added dropwise to a suspension of sodium hydride (60% dispersion in mineral oil, 520 mg, 13 mmol) in tetrahydrofuran (40 mL) and the mixture was stirred at room temperature for 2 hours. 2-Chloro-6-methoxy-3-nitropyridine (2 g, 10.6 mmol) was then added and the mixture was stirred for a further 2 hours at room temperature. The reaction mixture was then concentrated in vacuo and the residue was partitioned between dichloromethane and water. The aqueous solution was extracted with dichloromethane (2×50 mL) and the combined organic solution was dried over sodium sulfate and concentrated in vacuo. Purification of the residue by column chromatography on silica gel, eluting with dichloromethane:methanol:0.88 ammonia, 100:0:0 to 96:4:0.4, afforded the title compound as a pale yellow solid in 49% yield, 1.46 g.

¹HNMR(400 MHz, CDCl₃) δ: 2.60-2.70(m, 4H), 2.87(t, 2H), 3.70-3.75(m, 4H), 3.98(s, 3H), 4.64(t, 2H), 6.37(d, 1H), 8.33 (d, 1H); LRMS ESI m/z 284 [M+H]⁺

Preparation 60

### 6-Methoxy-2-(2-morpholin-4-ylethoxy)pyridin-3-amine

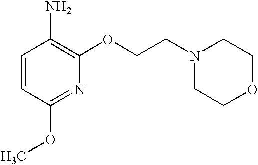

The title compound was prepared from the product of preparation 59, using the same method as that described for preparation 57, as a red oil in quantitative yield.

¹HNMR(400 MHz, CDCl₃) δ: 2.57-2.63(m, 4H), 2.83(t, 2H), 3.70-3.76(m, 4H), 3.82(s, 3H), 4.50(t,2H), 6.17(d, 1H), 6.95(d, 1H); LRMS APCI m/z 254 [M+H]⁺

1. A method of treating male sexual dysfunction, female sexual dysfunction, hypoactive sexual desire disorder, sexual arousal disorder, orgasmic disorder, sexual pain disorder, premature ejaculation, preterm labor, complications in labor, appetite disorder, feeding disorder, benign prostatic hyperplasia, premature birth, dysmenorrhoea, congestive heart failure, arterial hypertension, liver cirrhosis, nephrotic hypertension, ocular hypertension, obsessive compulsive disorder or neuropsychiatric disorder, the method comprising administering to a patient in need thereof a therapeutically effective amount of a compound of formula (I) 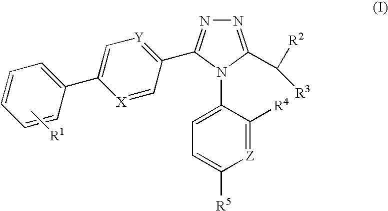
wherein:
X is C—R⁶ or N;
Y is C—R⁶ or N;
Z is C—R⁶ or N;
R¹ is selected from H, (C₁-C₆)alkyl, (C₁-C₆)alkoxy, halo, cyano, and C(O)NR⁷R⁸;
R² is selected from:
(i) H, OH, (C₁-C₆)alkoxy, OR⁹, NR⁷R⁸;
(ii) a 5-7 membered N-linked heterocycle containing 1-3 heteroatoms selected from N, O and S, optionally substituted with one to three groups selected from (C₁-C₆)alkyl, (C₁-C₆)alkoxy, oxo, and C(O)NR⁷R⁸; and
(iii) (C₁-C₆)alkyl optionally substituted with an N-linked 5-7 membered heterocycle containing 1-3 heteroatoms selected from N, O and S;
R³ is selected from H and (C₁-C₆)alkyl;
R⁴ is selected from H, (C₁-C₆)alkyl, (C₁-C₆)alkoxy, and OR⁹;
R⁵ is selected from H, halo, (C₁-C₆)alkyl, (C₁-C₆)alkoxy and NR⁷R⁸;
R⁶ is selected from H, halo, (C₁-C₆)alkyl, (C₁-C₆)alkoxy, cyano, NR⁷R⁸ and C(O)NR⁷R⁸;
R⁷ and R⁸, which may be the same or different, are selected from H, (C₁-C₆)alkyl, and C(O)R¹⁰;
R⁹ is (C₁-C₆)alkyl substituted with one to three groups selected from (C₁-C₆)alkoxy, NR⁷R⁸, and an N-linked 5-7 membered heterocycle containing 1-3 heteroatoms selected from N, O and S;
R¹⁰ is selected from (C₁-C₆)alkyl and (C₁-C₆)alkoxy;
or a tautomer thereof or a pharmaceutically acceptable salt, or polymorph of said compound or tautomer.

2. The method according to claim 1, wherein:
X is C—H;
Y is C—R⁶ or N;
Z is C—H or N;
R¹ is selected from (C₁-C₆)alkyl, (C₁-C₆)alkoxy, cyano, and C(O)NR⁷R⁸;
R² is selected from:
(i) H, OH, (C₁-C₆)alkoxy; and
(ii) a 5-7 membered N-linked heterocycle containing 1-3 N atoms, optionally substituted with one to three groups selected from (C₁-C₆)alkyl, (C₁-C₆)alkoxy, oxo, and C(O)NR⁷R⁸;
R³ is H;
R⁴ is selected from H, (C₁-C₆)alkyl, (C₁-C₆)alkoxy, and OR⁹;
R⁵ is selected from halo, (C₁-C₆)alkyl, and (C₁-C₆)alkoxy;
R⁶ is selected from H, halo, (C₁-C₆)alkyl, (C₁-C₆)alkoxy, cyano, NR⁷R⁸ and C(O)NR⁷R⁸;
R⁷ and R⁸, which may be the same or different, are selected from H, (C₁-C₆)alkyl, and C(O)R¹⁰;
R⁹ is (C₁-C₆)alkyl substituted with to three groups selected from (C₁-C₆)alkoxy, NR⁷R⁸, and an N-linked 5-7 membered heterocycle containing at least one N atom and, optionally, an additional 1-2 heteroatoms selected from N, O and S; and
R¹⁰ is selected from (C₁-C₆)alkyl and (C₁-C₆)alkoxy;
or a tautomer thereof or a pharmaceutically acceptable salt, or polymorph of said compound or tautomer.

3. The method according to claim 1, wherein:
R¹ is selected from (C₁-C₄)alkyl and (C₁-C₄)alkoxy;
R² is selected from (i) H and (C₁-C₃)alkoxy; and (ii) a 5-6 membered N-linked heterocycle containing 1-3 N atoms, optionally substituted with one to three groups selected from (C₁-C₆)alkyl, (C₁-C₆)alkoxy, oxo, and C(O)NR⁷R⁸;
R⁴ is selected from H, methyl, and OR⁹;
R⁵ is selected from methyl and methoxy;
R⁶ is selected from H and methoxy; and
R⁷ and R⁸ are H;
or a tautomer thereof or a pharmaceutically acceptable salt or polymorph of said compound or tautomer.

4. The method according to claim 1, wherein:
R¹ is selected from methyl and methoxy;
R² is selected from (i) H and methoxy; and (ii) an N-linked heterocycle selected from imidazole, 1,2,3-triazole, pyrazole and pyrrolidine, each optionally substituted with one to three groups selected from (C₁-C₃)alkyl and C(O)NR⁷R⁸; and
R⁴ is selected from H and methyl;
or a tautomer thereof or a pharmaceutically acceptable salt or polymorph of said compound or tautomer.

5. The method according to claim 1, wherein the compound of formula (I) is selected from:
6-methoxy-3-[3-(2′-methoxybiphenyl-4-yl)-5-methyl-4H-1,2,4-triazol-4-yl]-2-methylpyridine;
3-(2′,3-dimethoxybiphenyl-4-yl)-4-(4-methoxyphenyl)-5-methyl-4H-1,2,4-triazole;
2-methoxy-5-[3-(methoxymethyl)-5-(2′-methylbiphenyl-4-yl)-4H-1,2,4-triazol-4-yl]pyridine;
2-{[5-(2′-methoxybiphenyl-4-yl)-4-(4-methylphenyl)-4H-1,2,4-triazol-3-yl]methyl}-2H-1,2,3-triazole;
6-methoxy-3-[3-(2′-methoxybiphenyl-4-yl)-5-methyl-4H-1,2,4-triazol-4-yl]-2-methylpyridine; and
2-methoxy-5-[3-[5-(2-methylphenyl)pyridin-2-yl]-5-(2H-1,2,3-triazol-2-ylmethyl)-4H-1,2,4-triazol-4-yl]pyridine;
or a pharmaceutically acceptable salt thereof.

14. A compound of formula (I), 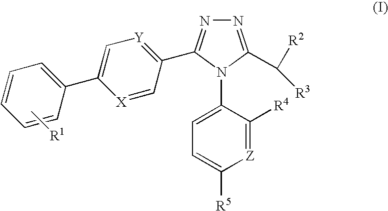
wherein:
X is C—R⁶ or N;
Y is C—R⁶ or N;
Z is C—R⁶ or N;
R¹ is selected from H, (C₁-C₆ alkyl, (C₁-C₆)alkoxy, halo, cyano, and C(O)NR⁷R⁸;
R² is selected from:
(i) H, OH, (C₁-C₆)alkoxy. OR⁹, NR⁷R⁸;
(ii) a 5-7 membered N-linked heterocycle containing 1-3 heteroatoms selected from N, O and S, optionally substituted with one to three groups selected from (C₁-C₆)alkyl, (C₁-C₆)alkoxy, oxo, and C(O)NR⁷R⁸; and
(iii) (C₁-C₆)alkyl optionally substituted with an N-linked 5-7 membered heterocycle containing 1-3 heteroatoms selected from N, O and S;
R³ is selected from H and (C₁-C₆)alkyl;
R⁴ is selected from H, (C₁-C₆)alkyl, (C₁-C₆)alkoxy, and OR⁹;
R⁵ is selected from H, halo, (C₁-C₆)alkyl, (C₁-C₆)alkoxy and NR⁷R⁸;
R⁶ is selected from H, halo, (C₁-C₆)alkyl, (C₁-C₆)alkoxy, cyano, NR⁷R⁸ and C(O)NR⁷R⁸;
R⁷ and R⁸, which may be the same or different, are selected from H, (C₁-C₆)alkyl, and C(O)R¹⁰;
R⁹ is (C₁-C₆)alkyl substituted with one to three groups selected from (C₁-C₆)alkoxy, NR⁷R⁸, and an N-linked 5-7 membered heterocycle containing 1-3 heteroatoms selected from N, O and S;
R¹⁰ is selected from (C₁-C₆)alkyl and (C₁-C₆)alkoxy;
or a tautomer thereof or a pharmaceutically acceptable salt or polymorph of said compound or tautomer.

15. A pharmaceutical composition comprising a compound of formula (I) as claimed in claim 14, or a pharmaceutically acceptable salt or polymorph thereof, and a pharmaceutically acceptable diluent or carrier.

16. A The pharmaceutical composition according to claim 15 further comprising one or more additional therapeutic agents.

18. The compound of formula (I) according to claim 14, wherein:
X is C—H;
Y is C—R⁶ or N;
Z is C—H or N;
R¹ is selected from (C₁-C₆)alkyl, (C₁-C₆)alkoxy, cyano, and C(O)NR⁷R⁸;
R² is selected from:
(i) H, OH, (C₁-C₆)alkoxy; and
(ii) a 5-7 membered N-linked heterocycle containing 1-3 N atoms, optionally substituted with one to three groups selected from (C₁-C₆)alkyl, (C₁-C₆)alkoxy, oxo, and C(O)NR⁷R⁸;
R³ is H;
R⁴ is selected from H, (C₁-C₆)alkyl, (C₁-C₆)alkoxy, and OR⁹;
R⁵ is selected from halo, (C₁-C₆)alkyl, and (C₁-C₆)alkoxy;
R⁶ is selected from H, halo, (C₁-C₆)alkyl, (C_{1-C}₆)alkoxy, cyano, NR⁷R⁸ and C(O)N⁷R⁸;
R⁷ and R⁸, which may be the same or different, are selected from H, (C₁-C₆)alkyl, and C(O)R¹⁰;
R⁹ is (C₁-C₆)alkyl substituted with one to three groups selected from (C₁-C₆)alkoxy, NR⁷R⁸, and an N-linked 5-7 membered heterocycle containing at least one N atom and, optionally, an additional 1-2 heteroatoms selected from N, O and S; and
R¹⁰ is selected from (C₁-C₆)alkyl and (C₁-C₆)alkoxy;
or a tautomer thereof or a pharmaceutically acceptable salt or polymorph of said compound or tautomer.

19. The compound of formula (I) according to claim 14, wherein:
R¹ is selected from (C₁-C₄)alkyl and (C₁-C₄)alkoxy;
R² is selected from (i) H and (C₁-C₃)alkoxy; and (ii) a 5-6 membered N-linked heterocycle containing 1-3 N atoms, optionally substituted with one to three groups selected from (C₁-C₆)alkyl, (C₁-C₆)alkoxy, oxo, and C(O)NR⁷R⁸;
R⁴is selected from H, methyl, and OR⁹;
R⁵ is selected from methyl and methoxy;
R⁶ is selected from H and methoxy; and
R⁷ and R⁸ are H;
or a tautomer thereof or a pharmaceutically acceptable salt or polymorph of said compound or tautomer.

20. The compound of formula (I) according to claim 14, wherein:
R¹ is selected from methyl and methoxy;
R² is selected from (i) H and methoxy; and (ii) an N-linked heterocycle selected from imidazole, 1,2,3-triazole, pyrazole and pyrrolidine, each optionally substituted with one to three groups selected from (C₁-C₃)alkyl and C(O)NR⁷R⁸; and
R⁴is selected from H and methyl;
or a tautomer thereof or a pharmaceutically acceptable salt or polymorph of said compound or tautomer.

21. 6-methoxy-3-[3-(2′-methoxybiphenyl-4-yl)-5-methyl-4H-1,2,4-triazol-4-yl]-2-methylpyridine;
3-(2′,3-dimethoxybiphenyl-4-yl)-4-(4-methoxyphenyl)-5-methyl-4H-1,2,4-triazole;
2-methoxy-5-[3-(methoxymethyl)-5-(2′-methylbiphenyl-4-yl)-4H-1,2,4-triazol-4-yl]pyridine;
2-{[5-(2′-methoxybiphenyl-4-yl)-4-(4-methylphenyl)-4H-1,2,4-triazol-3-yl]methyl}-2H-1,2,3-triazole;
6-methoxy-3-[3-(2′-methoxybiphenyl-4-yl)-5-methyl-4H-1,2,4-triazol-4-yl]-2-methylpyridine; or
2-methoxy-5-[3-[5-(2-methylphenyl)pyridin-2-yl]-5-(2H-1,2,3-triazol-2-ylmethyl)-4H-1,2,4-triazol-4-yl]pyridine;
or a pharmaceutically acceptable salt thereof.
